SEQUENCE LISTING (57 sequences)

SEQ ID NO: 1 - protein, 8 aa, Artificial Sequence
  Synthetic
SIYRYYGL

SEQ ID NO: 2 - protein, 15 aa, Artificial Sequence
  Synthetic
CASSTPGQGNERLFF

SEQ ID NO: 3 - protein, 14 aa, Artificial Sequence
  Synthetic
CASGGTGGQDTQYF

SEQ ID NO: 4 - protein, 14 aa, Artificial Sequence
  Synthetic
CASSFSGGAAEQFF

SEQ ID NO: 5 - protein, 14 aa, Artificial Sequence
  Synthetic
CASSFTGVGNTLYF

SEQ ID NO: 6 - protein, 14 aa, Artificial Sequence
  Synthetic
CASSGGGLAETLYF

SEQ ID NO: 7 - protein, 13 aa, Artificial Sequence
  Synthetic
CASSLGGRAEQFF

SEQ ID NO: 8 - protein, 14 aa, Artificial Sequence
  Synthetic
CASSLGVNNQAPLF

SEQ ID NO: 9 - protein, 15 aa, Artificial Sequence
  Synthetic
CASSHGGTSAETLYF

SEQ ID NO: 10 - protein, 15 aa, Artificial Sequence
  Synthetic
CASGDPGLGVGEQFF

SEQ ID NO: 11 - protein, 15 aa, Artificial Sequence
  Synthetic
CASSDGQGASETLYF

SEQ ID NO: 12 - protein, 12 aa, Artificial Sequence
  Synthetic
CASSYRGREQYF

SEQ ID NO: 13 - protein, 15 aa, Artificial Sequence
  Synthetic
CAWSLPWGAQNTLYF

SEQ ID NO: 14 - protein, 12 aa, Artificial Sequence
  Synthetic
CASSLRGREQYF

SEQ ID NO: 15 - protein, 14 aa, Artificial Sequence
  Synthetic
CASSADRGKNTLYF

SEQ ID NO: 16 - protein, 14 aa, Artificial Sequence
  Synthetic
CASGETNSGNTLYF

SEQ ID NO: 17 - protein, 13 aa, Artificial Sequence
  Synthetic
CASSFTGYAEQFF

SEQ ID NO: 18 - protein, 13 aa, Artificial Sequence
  Synthetic
CASKGGGEDTQYF

SEQ ID NO: 19 - protein, 14 aa, Artificial Sequence
  Synthetic
CASRPGTGSYEQYF

SEQ ID NO: 20 - protein, 13 aa, Artificial Sequence
  Synthetic
CASSDGNYAEQFF

SEQ ID NO: 21 - protein, 12 aa, Artificial Sequence
  Synthetic
CASSEDQNTLYF

SEQ ID NO: 22 - protein, 14 aa, Artificial Sequence
  Synthetic
CASSLGGANSPLYF

SEQ ID NO: 23 - protein, 13 aa, Artificial Sequence
  Synthetic
CASSSGQNTEVFF

SEQ ID NO: 24 - protein, 16 aa, Artificial Sequence
  Synthetic
CASSITGKEGQNTLYF

SEQ ID NO: 25 - protein, 12 aa, Artificial Sequence
  Synthetic
CARDREDQAPLF

SEQ ID NO: 26 - protein, 13 aa, Artificial Sequence
  Synthetic
CASSRWGQDTQYF

SEQ ID NO: 27 - protein, 14 aa, Artificial Sequence
  Synthetic
CSAGTGQNTGQLYF

SEQ ID NO: 28 - protein, 12 aa, Artificial Sequence
  Synthetic
CASAGDSYEQYF

SEQ ID NO: 29 - protein, 19 aa, Artificial Sequence
  Synthetic
CASSLTANSDYTFGSGTRL

SEQ ID NO: 30 - protein, 14 aa, Artificial Sequence
  Synthetic
CASGDARVEDTQYF

SEQ ID NO: 31 - protein, 12 aa, Artificial Sequence
  Synthetic
CAIQNSGNTLYF

SEQ ID NO: 32 - protein, 15 aa, Artificial Sequence
  Synthetic
CASSPGLGGAETLYF

SEQ ID NO: 33 - protein, 14 aa, Artificial Sequence
  Synthetic
CASSSTGGAGEQYF

SEQ ID NO: 34 - protein, 14 aa, Artificial Sequence
  Synthetic
CASNPLNSGNTLYF

SEQ ID NO: 35 - protein, 13 aa, Artificial Sequence
  Synthetic
CASLDWSQNTLYF

SEQ ID NO: 36 - protein, 16 aa, Artificial Sequence
  Synthetic
CASSTPHRGSQNTLYF

SEQ ID NO: 37 - protein, 10 aa, Artificial Sequence
  Synthetic
CASSITEVFF

SEQ ID NO: 38 - protein, 18 aa, Artificial Sequence
  Synthetic
CASSLGGCDYTFGSVTRL

SEQ ID NO: 39 - protein, 15 aa, Artificial Sequence
  Synthetic
CASSLGNLNTGQLYF

SEQ ID NO: 40 - protein, 15 aa, Artificial Sequence
  Synthetic
CASSFPSANTGQLYF

SEQ ID NO: 41 - protein, 15 aa, Artificial Sequence
  Synthetic
CASSLETGGTYEQYF

SEQ ID NO: 42 - protein, 14 aa, Artificial Sequence
  Synthetic
CAWRGTGVAETLYF

SEQ ID NO: 43 - protein, 13 aa, Artificial Sequence
  Synthetic
CASGDQNYAEQFF

SEQ ID NO: 44 - protein, 13 aa, Artificial Sequence
  Synthetic
CASSRDRVYEQYF

SEQ ID NO: 45 - protein, 15 aa, Artificial Sequence
  Synthetic
CASGDAGRGGNTLYF

SEQ ID NO: 46 - protein, 14 aa, Artificial Sequence
  Synthetic
CASSLEGGNTEVFF

SEQ ID NO: 47 - protein, 13 aa, Artificial Sequence
  Synthetic
CASSLDGGQEQYF

SEQ ID NO: 48 - protein, 14 aa, Artificial Sequence
  Synthetic
CASSFGGGTGQLYF

SEQ ID NO: 49 - protein, 14 aa, Artificial Sequence
  Synthetic
CASSLGQANTEVFF

SEQ ID NO: 50 - protein, 14 aa, Artificial Sequence
  Synthetic
CASRVQGGAETLYF

SEQ ID NO: 51 - protein, 14 aa, Artificial Sequence
  Synthetic
CASSARGLEDTQYF

SEQ ID NO: 52 - protein, 13 aa, Artificial Sequence
  Synthetic
CASSPTGFAEQFF

SEQ ID NO: 53 - protein, 13 aa, Artificial Sequence
  Synthetic
CASSFWGEDTQYF

SEQ ID NO: 54 - protein, 17 aa, Artificial Sequence
  Synthetic
CASSISGDYTFGSVTRL

SEQ ID NO: 55 - protein, 15 aa, Artificial Sequence
  Synthetic
CASSLARRSGNTLYF

SEQ ID NO: 56 - protein, 19 aa, Artificial Sequence
  Synthetic
CASSFGLNSDYTFGSVTRL

SEQ ID NO: 57 - protein, 13 aa, Artificial Sequence
  Synthetic
CASSEGDQNTLYF

(19) United States
(12) Patent Application Publication
(10) Pub. No.: US 2024/0156911 A1
(43) Pub. Date: May 16, 2024

(54) LUNG-CANCER SPECIFIC T CELL DYSFUNCTION

(71) Applicant: Massachusetts Institute of Technology, Cambridge, MA (US)

(72) Inventors: Karl Dane Wittrup, Chestnut Hill, MA (US); J. Christopher Love, Somerville, MA (US); Stefani Spranger, Boston, MA (US); Brendan Horton, Boston, MA (US); Duncan Morgan, Cambridge, MA (US)

(73) Assignee: Massachusetts Institute of Technology, Cambriged, MA (US)

(21) Appl. No.: 18/280,775

(22) PCT Filed: Mar. 7, 2022
(86) PCT No.: PCT/US2022/019156

§ 371 Date: Sep. 7, 2023

Related U.S. Application Data
(60) Provisional application No. 63/158,225, filed on Mar. 8, 2021.

(51) Int. Cl. A61K 38/20; A61K 39/00; A61P 35/00; C07K 16/28; C12Q 1/6886; G01N 33/569; G01N 33/574

(52) U.S. Cl. CPC A61K 38/2013; A61K 38/208; A61P 35/00; C07K 16/2818; C07K 16/2827; C12Q 1/6886; G01N 33/56972; G01N 33/57423; A61K 2039/505; C12Q 2600/158; G01N 2333/521; G01N 2333/70596

(57) ABSTRACT

Methods for identifying patients susceptible to treatment with checkpoint inhibitors are provided herein. Also provided are reagents for analyzing T cell responses for understanding resistance to checkpoint inhibitor therapy and understanding therapeutics for overcoming that resistance. Therapeutic strategies, for instance, for treating cancer, are also provided.

### RELATED APPLICATIONS

This application claims the benefit of the filing date under 35 U.S.C. § 119 of U.S. Provisional Application Ser. No. 63/158,225, filed Mar. 8, 2021, the entire contents of which are incorporated by reference herein.

### BACKGROUND INFORMATION

Cancer research has recently focused on the influence of the tumor microenvironment on disease progression, with increasing acknowledgment of an important role played by the immune system. Immune evasion during tumor progression is a hallmark of cancer. Recent research, such as that with checkpoint inhibition therapies, has provided some advances in the modulation of the interactions between tumors in the immune system. Checkpoint blockade therapy (CBT) involves releasing negative controls on T cell function to allow for effective immune responses to be developed against tumors. In the absence of this therapy the negative controls prevent T cells from developing an active immune response against the tumor. Clinical trials investigating these therapies have demonstrated dramatic and durable tumor regression across several different types of cancer, demonstrating the promise of immunotherapeutic strategies. Interestingly, however, only a fraction of patients undergoing these therapies experience these dramatic types of responses.

T cell dysfunction is a major mechanism for immune evasion in cancer. T cell exhaustion, one type of T cell dysfunction, occurs when effector T cells encounter persistent antigen. Exhaustion is characterized by a progressive loss of T cell effector function and highly expressed levels of inhibitory receptors. CBT reinvigorates anti-tumor T cell responses by blocking inhibitory receptors on exhausted CD8⁺ tumor-reactive T cells. CBT treatments have the potential to induce long-term clinical benefit in a fraction of patients and responses are frequently associated with the presence of CD8⁺ T cells in the tumor. In non-small cell lung cancer (NSCLC), however, over 60% of patients have evidence of T cell infiltration, but only about 30% respond to CBT.

These observations indicate that additional immune evasion mechanisms other than conventional T cell exhaustion may cause resistance to CBT in patients with T cell-infiltrated NSCLC. This phenotype is often referred to as non-functional T cell response (Herbst Nature 2014).

### SUMMARY OF INVENTION

Response to checkpoint blockade therapy (CBT) is associated with a pre-existing CD8⁺ T cell infiltrate, yet only a fraction of T cell-infiltrated tumors are resistant to therapy. A novel T cell-intrinsic mechanism of immunotherapy resistance, having profound implications for cancer therapy and solutions thereto have been discovered and are disclosed herein.

Aspects of the invention relate to a method for identifying a T cell having a dysfunctional property, by isolating a T cell and determining the presence of effector molecules associated with the T cell, wherein when the T cell expresses high levels of CD49d and CCL5 and TOX in the absence of TIM3 the T cell is a dysfunctional CD8+T (T_{dys}) cell. In some embodiments the presence of effector molecules is determined by flow cytometry. In some embodiments the T cells are not associated with or have low levels of effector molecules CD25, GzmB and IFN-γ. In some embodiments the T cells express high levels of genes associated with persistence and inhibition of effector T cell differentiation. In some embodiments the genes associated with persistence and inhibition of effector T cell differentiation are Sell, Pecam1, Bc16, Id3, Lef1, Ctla4, Bach2, Tox, and Tox2. In some embodiments the T cells express high levels of genes associated with lung T cell responses. In some embodiments the genes associated with lung T cell responses are Cc15, Itga4, Itgb1 and TOX in the absence of TIM3. In some embodiments the T cells express low levels or no genes associated with effector function and exhaustion. In some embodiments the genes associated with effector function are Gzma, Gzmb, Il2ra, Il12rb1, IL12rb2, Prdm1, Bhlhe40, and Id2 and exhaustion are Pdcd1, Havcr2, Tnfrsf4, and Cd160.

In other aspects, a composition comprising an isolated population of dysfunctional CD8⁺ T (T_{dys}) cells, wherein the T cells express high levels of CD49d and CCL5 and are not associated with or have low levels of effector molecules CD25, GzmB and IFN-γ is provided. In some embodiments the isolated population of T_{dys} cells are identified according to a method of any one of the methods disclosed herein. In some embodiments at least 80% of the cells in the population are T_{dys} cells.

A method for treating a subject having cancer is provided according to aspects of the invention. The method involves isolating a sample containing T cells from the subject, determining whether the sample contains dysfunctional CD8⁺ T (T_{dys}) cells, wherein if the sample contains T_{dys} cells then the subject is treated with a checkpoint based enhancing therapy and wherein if the sample does not contain T_{dys} cells then the subject is treated with a checkpoint based therapy (CBT). In some embodiments the T_{dys} are T_{Ldys} and the cancer is a lung cancer. In some embodiments the cancer is a melanoma. In some embodiments the cells in the sample are identified as T_{dys} cells according to a method as disclosed herein.

In some aspects the invention is a method for treating a subject having cancer, by administering to the subject a checkpoint based enhancing therapy, wherein the checkpoint based enhancing therapy is a compound that disrupts the activity of T_{dys} cells and administering to the subject a checkpoint based therapy (CBT) in an effective amount to treat the cancer. In some embodiments the compound that disrupts the activity of T_{dys} cells is a cytokine. In some embodiments the compound that disrupts the activity of T_{dys} cells is a promoter of the IL2 pathway. In some embodiments the compound that disrupts the activity of T_{dys} cells is a promoter of the IL12 pathway. In some embodiments the compound that disrupts the activity of T_{dys} cells is a promoter of the IL2 and IL12 pathway. In some embodiments the CBT is an inhibitory checkpoint antibody. In some embodiments inhibitory checkpoint antibody is selected from the group consisting of anti-PD1, anti-PD-L1, anti-CTLA4 therapy.

These and other aspects of the invention, as well as various embodiments thereof, will become more apparent in reference to the drawings and detailed description of the invention.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1G show KP tumors in the lung are resistant to CBT. FIG. 1A shows a scheme of tumor inoculation and CBT treatment. FIG. 1B shows outgrowth of flank KP tumors treated with CBT or control (control n=9, CBT n=9). FIGS. 1C-1D show representative example (as shown in FIG. 1C) and quantification (as shown in FIG. 1D) of lung tumor burden at day 21 in control and CBT treated mice assessed by H&E stain (control n=9, CBT n=9). FIG. 1E shows comparison between CBT efficacy in lung and flank tumors as percent change over control treatment (lung n=9, flank n=9). FIGS. 1F-1G show amount of CD8⁺ T cells in control and CBT treated lung and flank KP tumors at day 21 determined by IHC (all conditions n=9). Data are shown as mean±SEM and statistical analysis was conducted using a two-way Annova (b) or MWU test (d, e, g) with ***p<0.001; ****p<0.0001.

FIGS. 2A-2E show CD8⁺ lung TIL do not acquire effector or exhausted phenotypes observed in flank TIL. UMAP plots of single cell sequencing indicating treatment status and tumor location were developed and curated clusters were analyzed for gene expression. FIG. 2A shows contributions to each curated cluster based on treatment condition and tumor location. FIG. 2B shows volcano plot of DEGs between lung and flank CD8⁺ T cells. p-values were calculated using a two-sided Wilcoxon rank-sum test and were corrected with Bonferroni correction. Selected genes expressed significantly higher in lung (left) or flank (right) TIL are highlighted. FIG. 2C shows enrichment of DEG (lung is left; flank is right) in gene modules of a previously published data set on T cell activation states FDR q-values were calculated using a one-tailed hypergeometric test and were corrected with Bonferroni correction. FIG. 2D shows pathway analysis of DEGs showing enriched MSigDb hallmarks pathways for lung (left) and flank (right) CD8⁺ TIL. P-values were calculated using a two-tailed Wilcoxon rank-sum test and were corrected with Bonferroni correction as shown in FIG. 2D. FIG. 2E shows validation of differentially expressed key effector/exhaustion markers on lung and flank CD8⁺ TIL (lung n=6, flank n=6). Data are shown as mean±SEM and statistical analysis was conducted using a MWU test (as shown in FIG. 2E) with *p<0.05; **p<0.01, or as indicated in the methods section (as shown in FIGS. 2A-2D).

FIGS. 3A-3E shows CD8⁺ T cells primed in the mesenteric LN fail to acquire an effector phenotype. FIG. 3A shows schematic of experimental setup. FIG. 3B shows volcano plot of DEGs between 2C T cells primed in the mLN and iLN of tumor bearing mice isolated at 72 h post adoptive transfer. FIGS. 3C-3E show representative example and mean±SEM of CD25 (as shown in FIG. 3C), GzmB (as shown in FIG. 3D), and CD49d (as shown in FIG. 3E) expression levels on 2C T cells primed in the mLN and iLN of tumor bearing mice at 72 h post adoptive transfer (mLN n=6, iLN n=6). Statistical analysis was conducted using a MWU test (as shown in FIGS. 3C-3E) with **p<0.01 or as indicated in the methods section (as shown in FIG. 3B).

FIGS. 4A-4E show lung-specific CD8⁺ T cell dysfunction occurs in human NSCLC patients and can be overcome by combined IL-2 and IL-12 therapy. Three human data sets were analyzed for expression of exhaustion and T_{Ldys} signatures. FIG. 4A shows proportions of CD8⁺ T cells from individual patients that have exhaustion (light grey) or lung-specific T_{Ldys} (dark grey) signatures. FIG. 4B shows schematic of experimental design. FIG. 4C shows percentage of CD25 (left) and Gzmb (right) on 2C T cells in mLN following control or cytokine treatments (all conditions n=6). FIG. 4D shows schematic of experiment combining CBT with MSA therapy. FIG. 4E shows lung tumor burden assessed at day 21 following CBT, cytokine or combination therapy with FIG. 4E showing the quantification as mean±SEM (control and CBT n=5, MSA and CBT+MSA n=6)). Longitudinal μCT scans of a CBT+MSA treated mouse showing reduction of a lung tumor lesion over time (n=2) were performed (data not shown). Statistical analysis was conducted using a MWU test (as shown in FIGS. 4C and 4D) with *p<0.05, **p<0.01; or as indicated in the methods section (as shown in FIG. 4B).

FIGS. 5A-5F show differences between lung and flank CD8⁺ TIL are not driven by treatment status or TCR clones. FIG. 5A shows make up of annotated clusters for individual lung and flank tumors, control and CBT. FIG. 5B shows IL-2/STAT5 pathway genes differentially expressed by CD8⁺ c1 (left side and) and CD8⁺ c2 (dark gray). c) Flow cytometry of CD8⁺ TIL in LL/2 tumors. Lung n=6 flank n=5. Data are shown as mean+/− SEM. Statistical analysis was conducted using a MWU test with *p<0.05, **p<0.01 d) clonal expansion of T cell subsets in lung and flank tumors. e) Clonotypes recovered from flank and lung tumors (SEQ ID NOs: 2-57, in descending order, with SEQ ID NO. 2 referring to the top peptide sequence-CASSTPGQGNERLFF and SEQ ID NO. 57 referring to the bottom peptide sequence-CASSEGDQNTLYF). f) T cell specificity groups from individual lung and flank tumors, control and CBT treated.

FIG. 6 shows differences between lung and flank CD8⁺ TIL are not driven by tumor-intrinsic factors. A UMAP plot of scRNA-seq of KP.zsGreen tumor cells isolated from lung and flank tumors was generated (data not shown). FIG. 6 shows pathway enrichment analysis comparing KP.zsGreen tumor cells isolated from lung and flank tumors. Flank data is marked with asterisks.

FIGS. 7A-7D show T_{Ldys} is persistent T cell state. FIG. 7A shows tumor outgrowth of KP flank tumor in RAG2^{-/-} mice reconstituted with splenocytes (open), lung tumor TIL (filled dark gray), or flank tumor TIL (filled light gray) or not reconstituted (filled) (splenocytes n=3, lung TIL n=6, flank TIL n=9, not reconstituted n=3). FIG. 7B shows concomitant immunity assay measuring the tumor outgrowth of a secondary KP flank tumor in mice challenged 7-days prior with a primary flank (filled light grey) or lung KP (filled dark gray) tumors. Naïve mice served as control (open) (naive n=9, lung n=10, flank n=10). FIG. 7C shows concomitant immunity assay measuring the outgrowth of a second KP flank tumor in mice challenged 7 day prior with a primary flank tumor (filled circle) or no tumor (open circle), and in mice depleted of CD8⁺ T cells 48 hours before a first KP flank tumor (filled square) or no tumor (empty square). FIG. 7D shows concomitant immunity assay measuring lung weight on day 21 post intravenous re-challenge of mice bearing 7-day old flank tumor (light grey filled) or naïve mice (open) (naive n=6, flank n=6). Shown are mean±SEM, statistical analysis was conducted using two-way Annova (as shown in FIGS. 7A-7C) or a MWU test (as shown in FIG. 7D) with **p<0.01; ****p<0.0001.

FIGS. 8A-8E show blunted CD8⁺ T cell function against lung tumors is not due to lack of priming in the mLN. FIG. 8A shows flow cytometric analysis of endogenous SIY-reactive CD8⁺ T cells from lung and flank KP.SIY tumors (lung n=6, flank n=5).

FIG. 8B shows concomitant immunity assay measure tumor outgrowth of second KP.SIY flank tumors in naïve mice (open circle), or mice challenged 7 days prior with lung (top data line) or flank (bottom data line) KP.SIY tumors (all conditions n=6).

FIG. 8C shows number IFN-γ producing splenocytes assessed by IFN-γ ELISpot of lung or flank KP.SIY-bearing mice on day 7 post tumor challenge (naive n=3, lung n=9, flank n=9). d-e) Proliferation of CFSE-labeled, naïve 2C T cells adoptively transferred into tumor-bearing mice on day 7 post tumor engraftment and analyzed 72 hours later. Representative example (as shown in FIG. 8D) and mean±SEM (e) (mLN n=7, iLN n=8). Statistical analysis was conducted using a MWU test (as shown in FIG. 8A, 8C) or two-way ANOVA (as shown in FIG. 8B) with **p<0.01; ***p<0.001 (fill in all).

FIGS. 9A-9F show T_{Ldys} differentiation occurs during priming in the mLN. FIG. 9A shows enrichment of DEG (left lung; right flank) in gene modules of a previously published data set on T cell activation states. FDR q-values were calculated using a one-tailed hypergeometric test and were corrected with Bonferroni correction. FIG. 9B shows comparison between DEGs of recently primed 2C T cells (day 3, LN) and DEGs from the scRNA seq. TIL analysis (day 14, tumor). FIGS. 9C-9D show intracellular IFN-γ (as shown in FIG. 9C) and CCL5 (as shown in FIG. 9D) in adoptively transferred 2C T cells 72 h post transfer into flank and lung tumor bearing host mice (mLN n=3 iLN n=3). FIGS. 9E-9F show CD25 (FIG. 9E) and GzmB (FIG. 9F) expression on endogenous CD8⁺ T cells primed in mLN and iLN (mLN n=6, iLN n=6). Shown are mean±SEM, statistical analysis was conducted using a MWU test with **p<0.01.

FIGS. 10A-10B show MSA-IL2 and MSA-IL12 therapy enhances effector T cell differentiation against lung tumors. FIGS. 10A-10B show CD25 (FIG. 10A) and GzmB (FIG. 10B) expression on endogenous, polyclonal CD8⁺ T cells in mLN (top) and spleen (bottom) of lung tumor-bearing mice (Control, MSA-IL2, MSA-IL12 n=6, MSA-IL2+MSA-IL12 n=7). Shown are mean±SEM, statistical analysis was conducted using a MWU test with ***p<0.001; ****p<0.0001.

FIG. 11 shows longitudinal CT analysis of lung tumor control by CBT+MSA therapy. Representative CT scans of individual mice at the indicated time points post-tumor inoculation. Control n=1, CBT n=1, MSA+CBT n=2. Asterisks next to arrow indicate cropped tumor lesion.

FIG. 12 shows a schematic summary. Left: In response to flank tumors, tumor-reactive CD8⁺ T cells primed in the iLN upregulate CD25 and IL-12R, express effector molecules, show signs of conventional exhaustion, and respond to CBT. Right: In response to lung tumors, however, tumor-reactive CD8⁺ T cells primed in the mLN activate a lung-specific dysfunctional program (Tuy s), do not upregulate CD25 and IL-12R, fail to gain effector molecule expression, and do not acquire a conventional T cell exhaustion phenotype. CD8⁺ T_{Ldys} cells do not respond to CBT, suggesting that differentiation of CD8⁺ T cells into dysfunctional states other than conventional T cell exhaustion drives CBT resistance in a subset of T cell-infiltrated NSCLCs. Created with Biorender.com.

### DETAILED DESCRIPTION

The invention, in some aspects, relates to the identification of a class of cells responsible for disrupting the effectiveness of checkpoint blockade therapy (CBT) in the treatment of cancer. It is not clear why a significant number of cancer patients are resistant to CBT. One advance of the current invention relates to the discovery that a class of CD8⁺ T cells in this group of resistant patients is responsible for disrupting the effects of CBT. A novel state of lung-specific T cell dysfunction (T_{Ldys}) that is unresponsive to current CBT agents was identified according to the invention. A key finding of the studies described herein is that T_{Ldys} is induced during T cell priming in the TdLN, after which the activated tumor-reactive T cells fail to acquire effector function and markers of T cell exhaustion. This functionally impaired T cell response is found in the CD8⁺ TIL population, which fails to respond to anti-CTLA-4 and anti-PD-L1 CBT. It was discovered, for instance, in a lung cancer model that the populations of CD8⁺ T cells found in lung and flank tumors have different properties (dysfunctional (T_{Ldys}) or exhaustive respectively (Tex)) as a result of alternate differentiation pathways depending on the anatomic site of tumor growth. In experiments described in the Examples, flank T cells were shown to result in lower tumor growth compared to lung cells, suggesting that once induced, T_{Ldys} is a persistent T cell state that does not require the lung tumor microenvironment for maintenance. The data suggested that T_{Ldys} is a persistent dysfunctional T cell state established early during the anti-lung tumor immune response. T_{Ldys} are fundamentally distinct from conventional exhaustion, Tex. The T_{Ldys} can thus prevent CD8⁺ T cell responses against lung tumor lesion from responding to CBT due to T_{Ldys} differentiation during priming, preventing the acquisition of effector and exhaustion molecules.

Additionally, therapeutics for reversing the effect of these T_{Ldys} cell populations have been identified. It was shown in the Examples that this disruption in the T cell exhaustion process can be therapeutically rescued by compounds such as cytokines, i.e., IL-2 and IL-12 therapy, providing clarity on targeting pathways for identifying additional therapeutics.

Thus, the invention teaches that patients with a predominantly dysfunctional, T_{Ldys}-like CD8⁺ T cell response fail to respond to CBT, while patients with a sizable fraction of exhausted T cells observe a clinical benefit in response to CBT. Responsiveness to CBT is determined during T cell priming in the TdLN, with T cells in CBT-resistant tumors differentiating into a lung-specific dysfunctional (T_{Ldys}) state distinct from conventional T cell exhaustion (Tex). The inability of CBT to rescue these dysfunctional, but not exhausted, T cells provides an explanation for failed responses to immunotherapy in T cell-inflamed tumors, both in NSCLC and other cancer types.

In some aspects, a genetically engineered cell line is provided. The cell line may be used for studying the mechanisms of immune evasion in the context of T cell infiltration in the lung. It may also be used for identifying therapeutics. The cell line is a syngeneic lung cancer cell line, derived from an autochthonous NSCLC model driven by KrasG12D and deletion of p53 (KP cell line). Inoculation of KP tumor cells by routes such as intravenous or subcutaneous injection can be used to establish lung tumor or control flank tumors. In some embodiments the cell is a tumor cell stably expressing cerulean-SIY, for instance, SIYRYYGL (SEQ ID NO. 1). Engineered KP cells expressing the model antigen SIYRYYGL (KP.SIY) (SEQ ID NO. 1) can be made for instance using a pLV-EFla-IRES-puro vector digested with BamHI and EcoRI restriction enzymes (NEB) to linearize the vector and adding a cerulean—SIY insert (generated using the Cerulean-N1 vector linked to a codon-optimized sequence of the SIYRYYGL (SIY) peptide (SEQ ID NO. 1)). The inserts can be cloned into a linearized pLV-EFla-IRES-puro vector (final construct referred to as ‘pLV-EF1α-cerulean-SIY-IRES-puro’) using the In-Fusion cloning kit, amplified, and sequenced for accuracy.

In some aspects, the invention is a method for treating a subject having cancer. The method may involve a step in which a subject is selected based on their susceptibility to therapeutic checkpoint based therapy (CBT). A subject may be determined to be susceptible to CBT by assessing the presence of dysfunctional T_{dys}-like CD8⁺ T cells. The presence of these cells can be determined by examining markers associated with these cells. For instance, CD8⁺ T cell sample may be obtained from the subject and markers may be examined.

A T_{dys} cell marker is any molecule which identifies, alone, or in combination with other molecules a CD8⁺ T cell as a T_{dys} cell. For instance, in some embodiments the T_{dys} cells are not associated with or have low levels of effector molecules CD25, GzmB and IFN-γ and higher levels of CD49d and CCL5. In some embodiments the T cells express high levels of genes associated with persistence and inhibition of effector T cell differentiation. In some embodiments the genes associated with persistence and inhibition of effector T cell differentiation are Sell, Pecam1, Bc16, Id3, Lef1, Ctla4, Bach2, Tox, and Tox2. In some embodiments the T cells express high levels of genes associated with lung T cell responses. In some embodiments the genes associated with lung T cell responses are CCL5, Itga4, Itgb1. In some embodiments the T cells express low levels or no genes associated with effector function and exhaustion. In some embodiments the genes associated with effector function are Gzma, Gzmb, Il2ra, Il12rb1, 1112rb2, Prdm1, Bhlhe40, and Id2 and exhaustion are Pdcd1, Havcr2, Tnfrsf4, and Cd160.

The presence or absence of specific markers can be determined using any known assays in the art, such as flow cytometry, immunofluorescence, or nucleic acid analysis techniques.

If a subject is determined to have dysfunctional T_{dys}-like CD8⁺ T cells the subject can be administered a checkpoint based enhancing therapy in order to make the subject susceptible to CBT. A checkpoint based enhancing therapy, as used herein is a compound that disrupts the activity of T_{dys} cells, enabling the T cell exhaustion pathway. For instance, the compound may promote cell signaling through IL2 and/or IL12 pathways. In some embodiments the compound that disrupts the activity of T_{dys} cells is a cytokine such as IL2 and/or IL12. In other embodiments the compound that disrupts the activity of T_{dys} cells is a promoter of the IL2 and/or IL12 pathway. A promoter of the IL2 and/or IL12 pathway may be, for instance, an agonist of IL2 and/or IL12 or a compound that activates a downstream factor in the pathway.

Once the T_{dys} cell activity is disrupted and the T cell exhaustion pathway is enabled, the subject can be treated with a CBT in order to enhance the anti-tumor response in the subject. The CBT may be, for instance, an inhibitory checkpoint antibody. Checkpoint inhibitors are a relatively new class of drugs which show a lot of promise in cancer therapy. The immune system has multiple checkpoints to avoid the overactivation of the immune system on healthy cells. By using the checkpoints, proteins that indicate a cell is healthy, tumor cells can escape detection by the immune system. Certain checkpoints have been studied as targets for cancer therapy. CTLA-4 is aberrantly upregulated and present on the surface of T cells in certain cancers, resulting in a diminished T cell reaction in response to the tumor cells. PD1, another checkpoint, has been found to be upregulated in certain tumors, inhibiting T cell function and allowing the tumor to evade the immune system. In response, checkpoint inhibitors have been developed to enhance the immune system's anti-tumor response. These molecules are designed to permit the immune system to “see” the abnormal cell markers, thus resulting in an appropriate immune response to the cancer cell. For example, ipilimumab, an FDA-approved checkpoint inhibitor, targets CTLA-4, a molecule present on the surface of T cells that prevents the immune system from over activating. Ipilimumab, an artificial antibody, blocks T cells from being slowed by CTLA-4. It is used in the treatment of melanoma. Other examples of checkpoint inhibitors undergoing testing include: pembrolizumab (PD-1 receptor inhibitor), nivolumab (PD-1 inhibitor), MPDL3280A (PD-L1 inhibitor), MEDI4736 (PD-L1 inhibitor), MEDI0680 (PD-1 inhibitor), and MSB0010718C (PD-L1 inhibitor).

The compounds and method disclosed herein relate to the treatment of cancer. The compounds and methods of the invention may be used alone or in combination with other anti-cancer therapies. When a therapy is used in combination with another therapy the combined therapies may be administered together at the same time in the same or a separate administration vehicle. Alternatively, the two or more therapies can be administered at different times.

The other anti-cancer therapies useful in combination with the molecules of the invention include but are not limited to T cell targeted therapies, a kinase inhibitor, chemotherapy, radiation, an immunotherapy, or cytokine therapy. T cell targeted therapies include but are not limited to BiTEs, adoptive T cell transfer therapy, chimerical antigen receptor (CAR) T cell therapy, cancer vaccines, and checkpoint blockade. Checkpoint blockade or checkpoint inhibitors include but are not limited to anti-PD1, anti-PD-L1, and anti-CTLA4 therapy.

Bi-specific T-cell engagers (BiTEs), a class of artificial monoclonal antibodies, link T cells and tumor cells and are used as anti-cancer drugs. The BiTE linkage allows the cytotoxic T cell to recognize a tumor's surface antigen, allowing the T cell to exert its cytotoxic effects without requiring the binding of its specific T cell receptor. The process then parallels the physiological process that occurs when T cells attack tumor cells; the T cells produce proteins, such as perforin and granzymes, which enter the tumor cell and ultimately result in its apoptosis. The entire process occurs independently of the presence of MHC I or other co-stimulatory molecules. BiTEs consist of two single-chain variable fragments (scFvs) of different antibodies from four different genes on a single peptide and are approximately 55 kilodaltons in size. One scFv binds to T cells via the CD3 receptor, while the other scFv binds to the tumor via a tumor-specific molecule. There are several clinical trials relating to BiTEs, including MT110, blinaturmomab (MT 103), BAY2010112, MEDI-565, and MEDI-538. Potential clinical applications include treatment of non-Hodgkin's lymphoma, acute lymphoblastic leukemia, gastrointestinal cancers, lung cancers, melanoma, and acute myeloid lymphoma.

Adoptive T cell transfer therapy is a treatment under which T cells are removed from a donor, expanded and possibly modified, and then transferred back into the same patient or into a new recipient host in an effort to transfer the immunologic functionality and characteristics of the transferred T cells into the new host. Essentially, the immunotherapy engineers the patients' own immune cells to recognize and attack their specific tumors; the therapy can be used to target specific viral antigens and non-viral tumor antigens. Autologous cells are generally used in order to minimize the risk of graft-versus-host disease. The T cells isolated from the donor are expanded in vitro, capitalizing on the immunomodulatory action of interleukin-2 and the proliferative qualities of anti-CD3, and possibly modified. Using gene modification, the T cells can be transduced with genes encoding different antigen receptors, including artificial chimeric T cell receptors, which recognize target antigens on tumor cell surfaces through a high affinity extracellular domain. Other groups have used T cells taken from the patient's blood after the administration of a cancer vaccine, finding that “primed” rare tumor antigen-specific T cells are easier to expand in culture. The T cells are then administered to the recipient patient in large numbers in an activated state. The number of T cells created is greater than that which would result from vaccination alone. Antigen-specific T cells expanded in vitro and then administered to patients have been shown to eradicate established tumors and reconstitute immunity against MCV and Epstein-Barr virus. Different forms of the therapy have been used as treatments for cancer; for example, culturing tumor-infiltrating lymphocytes (TILs), isolating and expanding a specific T cell or clone, and using T cells that have been engineered to potently recognize and attack specific tumors.

One type of adoptive T cell transfer includes the addition of chimeric antigen receptors (CARs) to the T cells to redirect the specificity of the cytotoxic and helper T cells. CARs combine target cell recognition in a non-MHC-restricted manner and immune effector function; the transformed T cells are able to recognize specific tumor antigens. T cells can be engineered to target nearly any tumor associated antigen. CAR T cells consist of an antigen-binding domain, usually a single-chain variable fragment derived from a monoclonal antibody, and one or more intracellular T cell signaling domains. By using antibody-mediated TAA recognition, thymic selection, MHC down-regulation, and altered peptide processing are avoided. In addition, antibody-antigen affinity is several times stronger than natural TCR-mediated recognition. The technology has been applied to target the CD19 antigen in patients with hematologic malignancies but has not shown great success in solid tumors, where researchers have found a high rate of on-target/off-target toxicity. First generation CAR T cells featured intracellular domains from the CD3 zeta chain, the primary transmitter of signals from endogenous TCRs. Second generation CAR T cells included intracellular signaling domains from various co-stimulatory protein receptors (CD28, 41BB, ICOS) to the cytoplasmic tail of the CAR, permitting additional signals to the T cell. This improved the antitumor activity of the T cells. Third generation CAR T cells include multiple signaling domains (CD3z-CD28-41BB, CD3z-CD28-0X40, and others) to further improve potency. CARs are being studied for the following malignancies: ovarian cancer, renal cell carcinoma, B-cell malignancies, ALL, B-ALL, leukemia, refractory follicular lymphoma, B-NHL, B-lineage lymphoid malignancies post-UCBT, CLL, lymphomas, mantle cell lymphoma, Hodgkin's lymphoma, AML, cervical carcinoma, breast cancer, colorectal cancer, prostate cancer, rhabdomyosarcoma, neuroblastoma, melanoma, medullobastoma, lung malignancies, osteosarcoma, glioblastoma, and tumor neovasculature.

Cancer vaccines stimulate or restore the patient's immune system's ability to fight cancer. Preventative (prophylactic) cancer vaccines can target infectious agents that cause or contribute to the development of cancer and use antigens to generate an immune response. In the US, Gardasil® and Cervarix® are FDA-approved preventative vaccines against the two predominant types of HPV. In addition, a vaccine for hepatitis B virus (HBV) infections was developed and approved in 1981. Most children are vaccinated against HBV shortly after birth. Therapeutic vaccines have been created to delay or halt cancer growth, cause tumor shrinkage, prevent cancer recurrence, or to eliminate cancer cells that have not been killed by other forms of treatment. The FDA has approved Provenge®, a cancer treatment vaccine, for use in some men with metastatic prostate cancer and Oncophage® was approved in Russia for the treatment of kidney cancer. There are active clinical trials for treatment vaccines with respect to at least the following cancers: bladder cancer, brain tumors, breast cancer, cervical cancer, Hodgkin lymphoma, kidney cancer, leukemia, lung cancer, melanoma, multiple myeloma, Non-Hodgkin lymphoma, pancreatic cancer, prostate cancer, and solid tumors.

A cancer vaccine is comprised of a cancer antigen and optionally an adjuvant or carrier. As used herein a cancer antigen is broadly defined as an antigen expressed by a cancer cell. Preferably, the antigen is expressed at the cell surface of the cancer cell. Even more preferably, the antigen is one which is not expressed by normal cells, or at least not expressed to the same level as in cancer cells. For example, some cancer antigens are normally silent (i.e., not expressed) in normal cells, some are expressed only at certain stages of differentiation and others are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. The differential expression of cancer antigens in normal and cancer cells can be exploited in order to target cancer cells. As used herein, the terms “cancer antigen” and “tumor antigen” are used interchangeably.

Antibody-based therapies may also be used as an anti-cancer therapy. Antibody therapies can be used as a delivery system for the specific targeting of toxic substances to cancer cells. Antibodies are usually conjugated to toxins such as ricin (e.g., from castor beans), calicheamicin and maytansinoids, to radioactive isotopes such as Iodine-131 and Yttrium-90, to chemotherapeutic agents (as described herein), or to biological response modifiers. In this way, the toxic substances can be concentrated in the region of the cancer and non-specific toxicity to normal cells can be minimized.

Antibodies which bind to vasculature, such as those which bind to endothelial cells, are also useful. Generally solid tumors are dependent upon newly formed blood vessels to survive, and thus most tumors are capable of recruiting and stimulating the growth of new blood vessels. As a result, one strategy of many cancer medicaments is to attack the blood vessels feeding a tumor and/or the connective tissues (or stroma) supporting such blood vessels.

Kinase inhibitors block the action of one or more kinases. Kinases add a phosphate group to a protein or other organic molecule, generally to the serine, threonine, or tyrosine amino acid. Because phosphorylation is a necessary step in some cancers, kinase inhibitors can be used as a drug to treat the disease. Tyrosine kinase inhibitors are a class of chemotherapy medications that specifically block the actions of tyrosine kinase either by blocking the phosphorylation or by inhibiting EGFRs to prevent uncontrolled cell growth. The following is a non-exhaustive list of FDA-approved kinase inhibitors: axitinib, bosutinib, cetuximab, crizotinib, dasatinib, erlotinib, fostamatinib, gefitinib, ibrutinib, imatinib, lapatinib, lenvatinib, mubritinib, nilotinib, pazopanib, pegaptanib, ruxolitinib, sorafenib, sunitinib, SU6656, vandetanib, and vemurafenib.

The cancer therapy referred to as NK Cell Transfer involves implantation of NK cells. The adoptive transfer of NK cells has yielded mixed results, and studies are being conducted with respect to acute myeloid leukemia, hematological malignancies, acute lymphoblastic leukemia, ovarian cancer, fallopian tube cancer, primary peritoneal cancer, neuroblastoma, lymphomas, colorectal cancer, NSCLC, Non-Hodgkin's lymphoma, Hodgkin's lymphoma, and solid tumors.

Examples of chemotherapeutic agents which can be used according to the invention include but are not limited to Aminoglutethimide, Asparaginase, Busulfan, Carboplatin, Chlorombucil, Cytarabine HCI, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate.

In one aspect, a method for treating cancer is provided which involves administering the compositions of the invention to a subject having cancer. A “subject having cancer” is a subject that has been diagnosed with a cancer. In some embodiments, the subject has a cancer type characterized by a solid mass tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography), or by needle aspirations. The use of these latter techniques is more common in early detection. Molecular and phenotypic analysis of cancer cells within a tissue will usually confirm if the cancer is endogenous to the tissue or if the lesion is due to metastasis from another site. In other embodiments the cancer may be a cancer of the blood such as leukemia or lymphoma.

A cancer cell is a cell that divides and reproduces abnormally due to a loss of normal growth control. Cancer cells almost always arise from at least one genetic mutation. In some instances, it is possible to distinguish cancer cells from their normal counterparts based on profiles of expressed genes and proteins, as well as to the level of their expression. Genes commonly affected in cancer cells include oncogenes, such as ras, neu/HER2/erbB, myb, myc and abl, as well as tumor suppressor genes such as p53, Rb, DCC, RET and WT. Cancer-related mutations in some of these genes leads to a decrease in their expression or a complete deletion. In others, mutations cause an increase in expression or the expression of an activated variant of the normal counterpart. Genetic mutations in cancer cells can be targets of anti-cancer therapies in some instances. For example, some medicaments target proteins which are thought to be necessary for cancer cell survival and division, such as cell cycle proteins (e.g., cyclin dependent kinases), telomerase and telomerase associated proteins, and tumor suppressor proteins, many of which are upregulated, or unregulated, in cancer cells.

There are two types of tumors, benign and malignant. Nearly all benign tumors are encapsulated and are noninvasive; in contrast, malignant tumors are almost never encapsulated but invade adjacent tissue by infiltrative destructive growth. This infiltrative growth can be followed by tumor cells implanting at sites discontinuous with the original tumor. The method of the invention can be used to treat cancers or tumors in humans, including but not limited to: sarcoma, carcinoma, fibroma, leukemia, lymphoma, melanoma, myeloma, neuroblastoma, rhabdomyosarcoma, retinoblastoma, and glioma as well as each of the other tumors described herein.

A metastasis is a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Cancers include, but are not limited to, basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; liver cancer; lung cancer (e.g. small cell and non-small cell (NSCLC)); lymphoma including Hodgkin's and Non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

The compounds are useful for treating cancer in a subject. A “subject” shall mean a human or vertebrate mammal including but not limited to a dog, cat, horse, cow, pig, sheep, goat, or primate, e.g., monkey.

The term “effective amount” of a compound refers to the amount necessary or sufficient to realize a desired biologic effect. For example, an effective amount of a compound disclosed herein could be that amount necessary to cause sensitization of T cells to CBT, resulting in a functional immune response against a tumor antigen.

In some instances, a sub-therapeutic dosage of either the compound or the anti-cancer therapy, or a sub-therapeutic dosage of both, is used in the treatment of a subject having, or at risk of developing, cancer. When the two classes of drugs are used together, the anti-cancer therapy may be administered in a sub-therapeutic dose and still produce a desirable therapeutic result. A “sub-therapeutic dose” as used herein refers to a dosage which is less than that dosage which would produce a therapeutic result in the subject if administered in the absence of the other agent. Thus, the sub-therapeutic dose of an anti-cancer therapy is one which would not produce the desired therapeutic result in the subject in the absence of the administration of the compound. Therapeutic doses of anti-cancer therapies are well known in the field of medicine for the treatment of cancer. These dosages have been extensively described in references such as Remington's Pharmaceutical Sciences; as well as many other medical references relied upon by the medical profession as guidance for the treatment of cancer.

For any compound described herein a therapeutically effective amount can be initially determined from cell culture assays. Those results can be used to determine an effective amount of the particular compound, the particular subject, and the dosage can be adjusted upwards or downwards to achieve the desired levels in the subject.

Therapeutically effective amounts can also be determined in animal studies. For instance, the effective amount of compound to treat a cancer can be assessed using in vivo assays of tumor regression and/or prevention of tumor formation and/or measurements of immune function and/or recruitment of immune cells into tumors. Relevant animal models include assays in which malignant cells are injected into the animal subjects, usually in a defined site. Relevant animal models may also include genetically engineered models. KP mice described herein are a relevant genetically engineered animal model of NSCLC. Generally, a range of compound doses are administered to the animal, optionally along with a range of anti-cancer therapy doses. Inhibition of the growth of a tumor following the injection of the malignant cells is indicative of the ability to reduce the risk of developing a cancer. Inhibition of further growth (or reduction in size) of a pre-existing tumor is indicative of the ability to treat the cancer. Increased infiltration of a tumor by immune cells is indicative of the ability to stimulate anti-cancer immune responses.

The applied dose of both the compound and other combined therapies can be adjusted based on the relative bioavailability and potency of the administered compounds. Adjusting the dose to achieve maximal efficacy based on the methods described above and other methods are well within the capabilities of the ordinarily skilled artisan. Subject doses of the compounds described herein typically range from about 0.1 μg to 10,000 μg, more typically from about 1 μg/day to 8000 μg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

The compounds may be administered a single time to a subject. Alternatively, the compounds may be administered multiple times, as needed, i.e. 2, 3, 4, 5, 6, 7, 8, 9, or 10 doses may be administered to a subject. In other embodiments of the invention, the compound is administered on a routine schedule. When the compound is administered with an anti-cancer therapy, the anti-cancer therapy may also be administered on a routine schedule, but alternatively, may be administered as symptoms arise. A “routine schedule” as used herein, refers to a predetermined designated period of time. The routine schedule may encompass periods of time which are identical or which differ in length, as long as the schedule is predetermined. For instance, the routine schedule may involve administration of the compound on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration of the compound on a daily basis for the first week, followed by a monthly basis for several months, and then every three months after that. Any particular combination would be covered by the routine schedule as long as it is determined ahead of time that the appropriate schedule involves administration on a certain day. The schedule may be adjusted as deemed important by medical professionals.

The methods and compositions of the invention aim to treat subjects having a cancer. As used herein, the treatment of such subjects therefore embraces treatment prior to and after the existence of a cancer. Treatment after a cancer has started aims to reduce, ameliorate or altogether eliminate the cancer, and/or its associated symptoms, or prevent it from becoming worse.

The compounds and anti-cancer therapy may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Another suitable compound for sustained release delivery is GELFOAM, a commercially available product consisting of modified collagen fibers. Alternatively, the active compounds may be in powder form for constitution with a suitable vehicle, e.g., sterile pyrogen-free water, before use.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The compound compositions and the anti-cancer therapy compositions can be administered on fixed schedules or in different temporal relationships to one another. The various combinations have many advantages over the prior art methods of treating cancer, particularly with regard to increased specific cancer toxicity and decreased non-specific toxicity to normal tissues.

Anti-cancer therapies can be administered by any ordinary route for administering medications. Depending upon the type of cancer to be treated, anti-cancer therapies and the compounds of the invention may be inhaled, ingested or administered by systemic routes. Systemic routes include oral and parenteral. Inhaled medications are preferred in some embodiments because of the direct delivery to the lung, particularly in lung cancer patients. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. Preferred routes of administration include but are not limited to oral, parenteral, intramuscular, intranasal, intratracheal, intrathecal, intravenous, inhalation, ocular, vaginal, and rectal.

For use in therapy, an effective amount of the compound can be administered to a subject by any mode that delivers the compound to the affected organ or tissue, or alternatively to the immune system. “Administering” the pharmaceutical composition of the present invention may be accomplished by any means known to the skilled artisan. Preferred routes of administration include but are not limited to oral, parenteral, intramuscular, intranasal, intratracheal, inhalation, ocular, vaginal, and rectal.

For oral administration, the compounds (anti-cancer therapy, and the other therapeutic agent) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in a conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms such as suspensions, solutions or emulsions in oily or aqueous vehicles, and may contain formulatory agents such as suspending, stabilizing and/or dispersing agents.

In still other embodiments of the invention, the compounds are provided in the intravenous solutions, bags and/or tubing used to deliver transfusions into cancer patients. The intravenous bags and tubing may be themselves be coated on their internal surfaces with compounds, or they may be impregnated with compounds during manufacture. Methods for manufacture of intravenous systems for the delivery of biologically active materials are known in the art. Examples include those described in U.S. Pat. Nos. 4,973,307, and 5,250,028, issued to Alza, Corp.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The phraseology and terminology used herein is for the purpose of description and should not be regarded as limiting. The use of “including,” “comprising,” “having,” “containing,” “involving,” and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

The present invention is further illustrated by the following Examples, which in no way should be construed as further limiting. The entire contents of all of the references (including literature references, issued patents, published patent applications, and co-pending patent applications) cited throughout this application are hereby expressly incorporated herein by reference.

### EXAMPLES

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

T cell dysfunction is a major mechanism for immune evasion in cancer^{3,4}. T cell exhaustion—one type of T cell dysfunction—occurs when effector T cells encounter persistent antigen; it is characterized by a progressive loss of T cell effector function and highly expressed levels of inhibitory receptors^{4,5}. Checkpoint blockade therapy (CBT) reinvigorates anti-tumor T cell responses by blocking inhibitory receptors on exhausted CD8⁺ tumor-reactive T cells^{6,7}. CBT treatments have the potential to induce long-term clinical benefit in a fraction of patients^{6,7} and responses are frequently associated with the presence of CD8⁺ T cells in the tumor s. In non-small cell lung cancer (NSCLC), however, 61% of patients have evidence of T cell infiltration⁸, but only 32% respond to CBT². These observations indicate that additional immune evasion mechanisms other than conventional T cell exhaustion may cause resistance to CBT in patients with T cell-infiltrated NSCLC.

In order to demonstrate aspects of the invention a mouse model of non-small cell lung cancer (NSCLC) in which tumors in the lung fail to respond to checkpoint blockade therapy but those in the flank remain sensitive to therapy, was used to reveal that lung tumor-infiltrating CD8⁺ T cells lacked expression of effector and exhaustion molecules despite clonal expansion. As shown in the Examples disclosed herein, this lung-specific T cell dysfunction gene expression pattern was established early during T cell priming in the mediastinal lymph node despite robust T cell activation. In contrast, T cells responding to flank tumors and activated in the inguinal lymph node rapidly presented genes associated with effector function and conventional T cell exhaustion. CD8⁺ T cells from patients with NSCLC exhibited an analogous set of genes associated with lung-specific T cell dysfunction and were distinct from conventionally exhausted T cells.

It was found that therapeutic administration of IL-2 and IL-12 restored effector differentiation during T cell priming in the mediastinal lymph node and restored control of the lung tumor. These findings propose a novel mechanism of immunotherapy resistance in T cell-infiltrated lung cancers, characterized by the induction of a lung-specific dysfunctional response during T cell priming, wherein T cells fail to gain effector molecules, do not enter conventional T cell exhaustion and do not respond to current checkpoint blockade therapies.

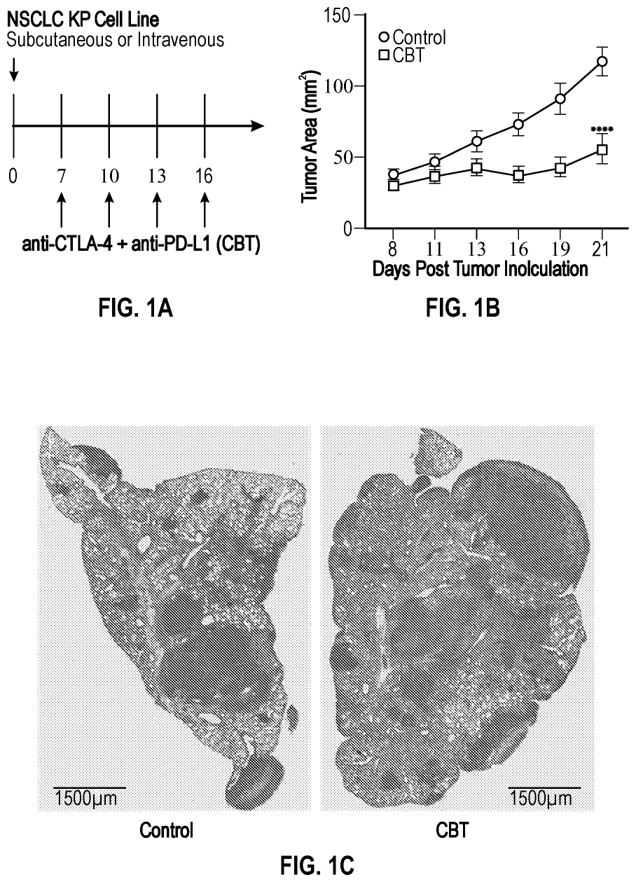

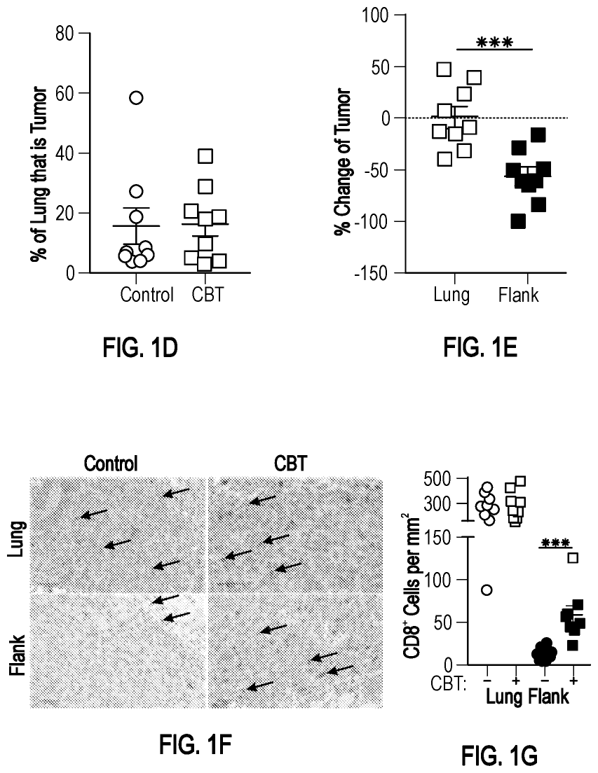

Lung tumors resist CBT To study mechanisms of immune evasion in the context of T cell infiltration in the lung, a syngeneic lung cancer cell line, derived from an autochthonous NSCLC model driven by Kras^{G12D} and deletion of p53 (KP cell line)⁹ was established. Inoculation of KP tumor cells via intravenous or subcutaneous injection established lung tumor or control flank tumors. To determine the responsiveness of lung and flank tumors to CBT, anti-CTLA-4 and anti-PD-L1 combination therapy was administered beginning on day 7 post tumor inoculation (FIG. 1A). Flank tumors had significantly reduced tumor growth in response to CBT (FIG. 1B), while tumor growth in the lung was unaffected by CBT (FIGS. 1C-1D). By calculating the relative response to CBT, it was confirmed that flank tumors responded significantly better than lung tumors (FIG. 1E). Poor response to CBT has been previously correlated with a lack of CD8⁺ T cell infiltration, both in pre-clinical models and patients^{1,10}. Therefore, the number of tumor-infiltrating CD8⁺ T cells (TIL) were assessed and identified a 21.3-fold higher number of CD8⁺ TIL in lung tumors compared to flank tumors (FIGS. 1F-1G). Consistent with previous reports^{1,10}, however, CD8⁺ TIL were more prevalent in flank tumors after CBT: A 4.5-fold increase was observed in CD8⁺ TIL density following therapy (FIGS. 1F-1G). In contrast, no change in CD8⁺ TIL density was observed in lung tumors after CBT (1.03-fold increase), consistent with the lack of tumor control (FIGS. 1F-1G). Therefore, the lack of response to CBT in lung tumors was not driven by a scarcity of tumor-infiltrating CD8⁺ T cells, but by other factors rendering lung tumor-reactive T cells insensitive to CBT.

Lung TIL are Dysfunctional but not Exhausted

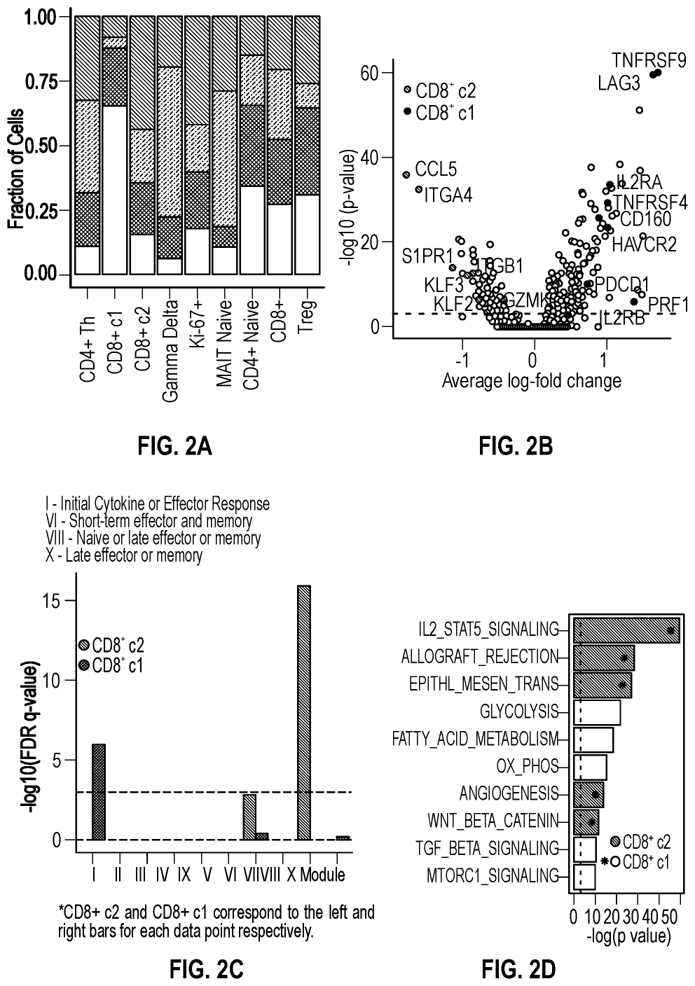

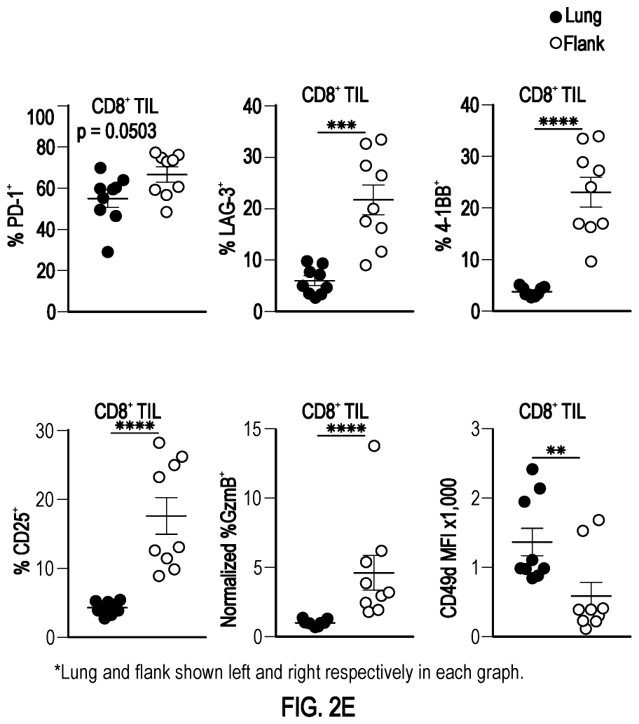

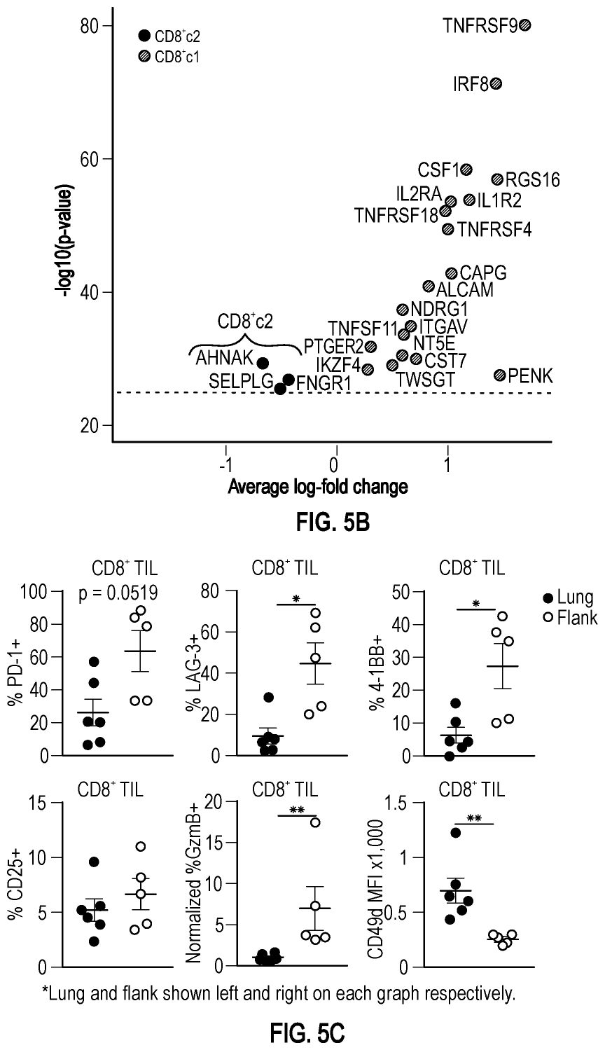

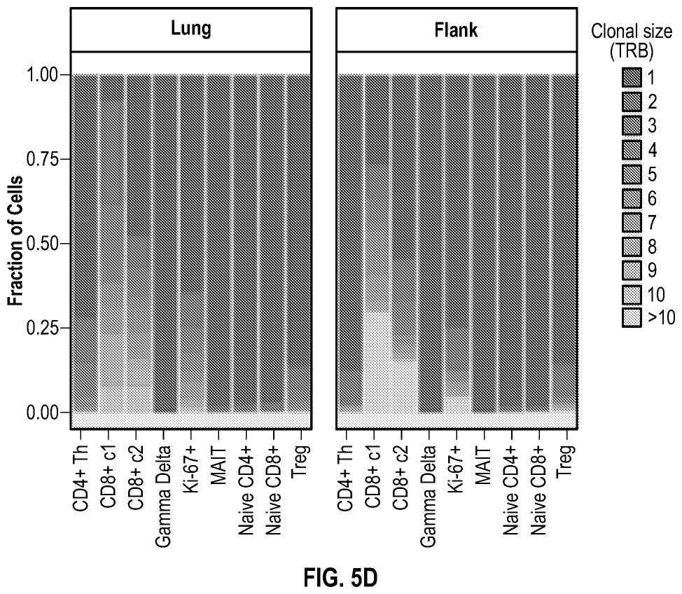

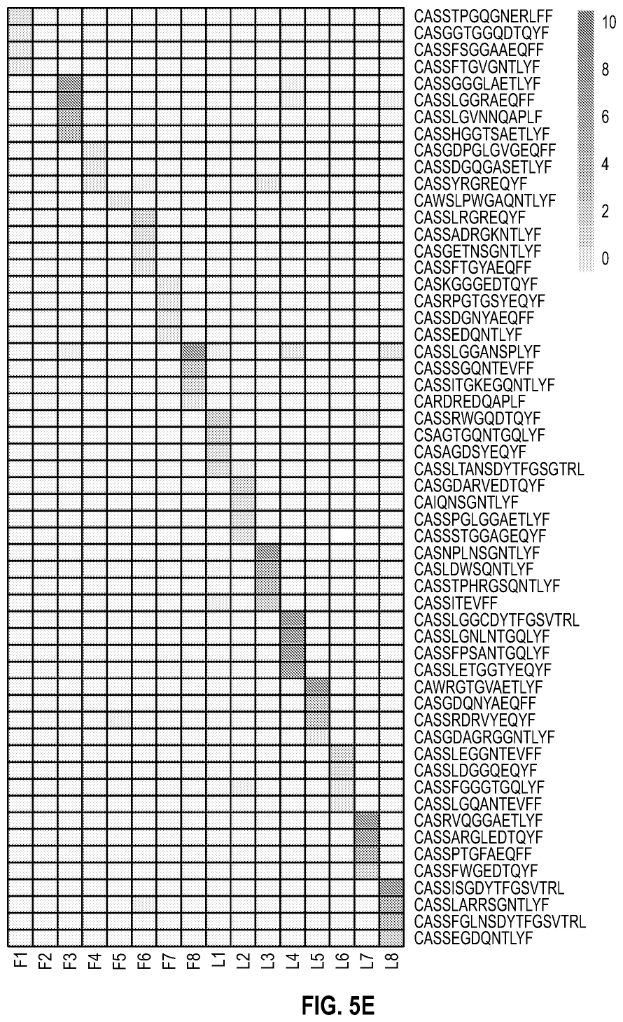

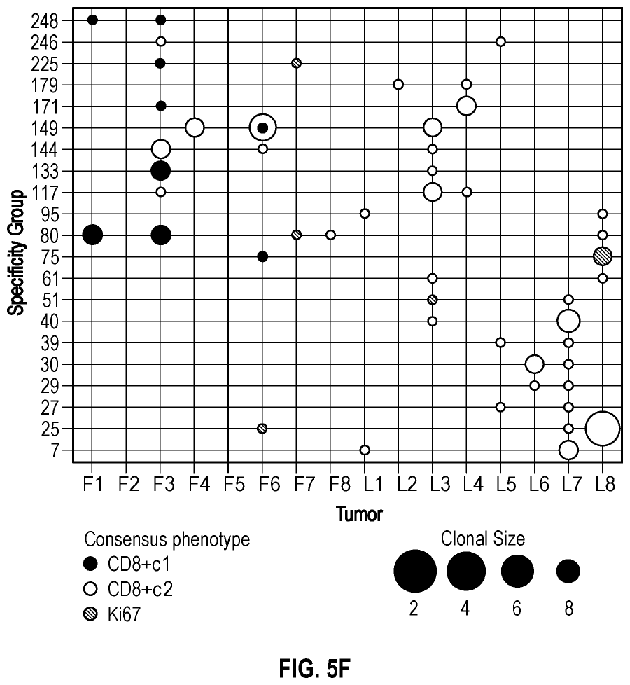

To determine the molecular underpinnings of CBT resistance in lung tumors, a single-cell RNA sequencing (scRNA-seq) was performed on tumor-infiltrating immune cells using Seq-Well¹¹. A total of 122,554 cells were sequences from eight flank and eight lung tumor-bearing mice on day 14 with and without CBT, including 10,774 T cells (data not shown). Nine distinct T cell phenotypes were identified, including multiple CD4⁺ and CD8⁺ T cell clusters, Tregs, MALT cells, γδT cell, and a proliferating Ki67⁺ cluster (data not shown). Cluster proportions were not significantly different between control and CBT conditions (FIG. 5A) but were present at different proportions depending on the tissue site of tumor growth (FIG. 2A). Given the importance of cytotoxic CD8⁺ effector T cells for the response to CBT and the eradication of tumor cells,^{1,12,13} Analysis was focused on both non-naïve CD8⁺ T cell clusters (CD8⁺ c1 and CD8⁺ c2). Differential gene expression analysis between CD8⁺ c1 and CD8⁺ c2 found that CD8⁺ c1, which was more prevalent in flank tumors, had increased transcripts of Pdcd1, Lag3, Tnfrsf9, Tnfrsf4, Havcr2, CD160, and Nrgn, all genes associated with conventional T cell exhaustion 14 (FIG. 2B). In contrast, CD8⁺ c2, which dominated the effector CD8⁺ T cell response in the lung, had significantly higher expression of Gzmk, Klf2, Klf3, S1pr1, and S1pr4 transcripts, which have been previously associated with a memory-like phenotype 15-18 (FIG. 2B). Higher transcript levels of Itga4, Itgb1 and Ccl5 were observed in CD8⁺ c2 (FIG. 2B), associated with lung homing^{19,21}. Gene module analysis and a direct comparison to published work affirmed that CD8⁺ c1 cells were effector or exhausted effector T cells (module I) while CD8⁺ c2 cells were of a memory phenotype (module VIII) (FIG. 2C)¹⁵. Pathways were determined differentially enriched between CD8⁺ c1 and CD8⁺ c2 and found IL-2/STAT5 signaling enriched in CD8⁺ c1, suggesting a correlation between T cell effector and exhaustion differentiation and IL-2 signaling (FIG. 2D). Consistently, transcripts involved in IL-2 signaling were upregulated in CD8⁺ c1 (FIG. 5B). These data suggested that two distinct CD8⁺ T cell responses were induced against the same KP tumor cell line depending on whether tumors grew in the flank or lung. CD8⁺ T cell responses against flank tumors were characterized by expression of molecules associated with effector functions and conventional T cell exhaustion, while CD8⁺ T cells in lung tumors lacked expression of effector molecules, appeared memory-like, and were distinct from conventionally exhausted T cell (Tₑₓ).

To validate key differentially expressed genes in CD8⁺ T cells on a protein level, flow cytometry was performed on TIL from lung or flank tumors. Despite differences in transcript levels, lung and flank TIL both expressed PD-1, indicative of antigen recognition occurring in both tumors²². Consistent with scRNA-seq, though, TIL from flank tumors showed significantly higher expression levels of molecules associated with T cell exhaustion, LAG-3 and 4-1BB, and effector differentiation, IL2RA (CD25) and Granzyme B (GzmB) (FIG. 2E). In contrast, CD49d (encoded by Itga4) was found to be expressed at higher levels on TIL from lung tumors (FIG. 2E). A second model of lung cancer (LL/2 cell line) provided similar results (FIG. 5C). Thus, the analysis provides evidence that lung TIL exhibit signs of T cell activation, yet lack hallmarks of effector T cell differentiation and do not resemble the conventional exhaustion phenotype. Rather, lung-tumor reactive TIL were uniquely dysfunctional, sharing transcriptional similarities to memory T cells but were unable to respond to CBT. This newly identified T cell state is referred to here as lung-specific T cell dysfunction or T_{Ldys}.

TCR Repertoire and Tumor Cells do not Drive T_{Ldys}

It was sought to determine differences between lung- and flank-specific T cell responses were driven by distinct TCR repertoires within lung and flank TIL. Tcra and Tcrb sequences were recovered from single-cell libraries²³. CD8⁺ c1, CD8⁺ c2 and Ki-67⁺ all showed evidence of clonal expansion, confirming that both sites of tumor growth induced an antigen-specific anti-tumor CD8⁺ T cell response (FIG. 5D). The majority of recovered clonotypes were private to any given mouse (FIG. 5E). To determine to what extent distinct TCR clonotypes may recognize similar antigens, Tcrb CDR3 sequences were used to define “specificity groups” of Tcrb that differ at most by one amino acid insertion, deletion, or substitution. A significant degree of similarity in specificity groups between flank and lung tumors were identified (FIG. 5F). This result allowed exclusion of drastically different T cell repertoires as the source for functional differences in the T cell response.

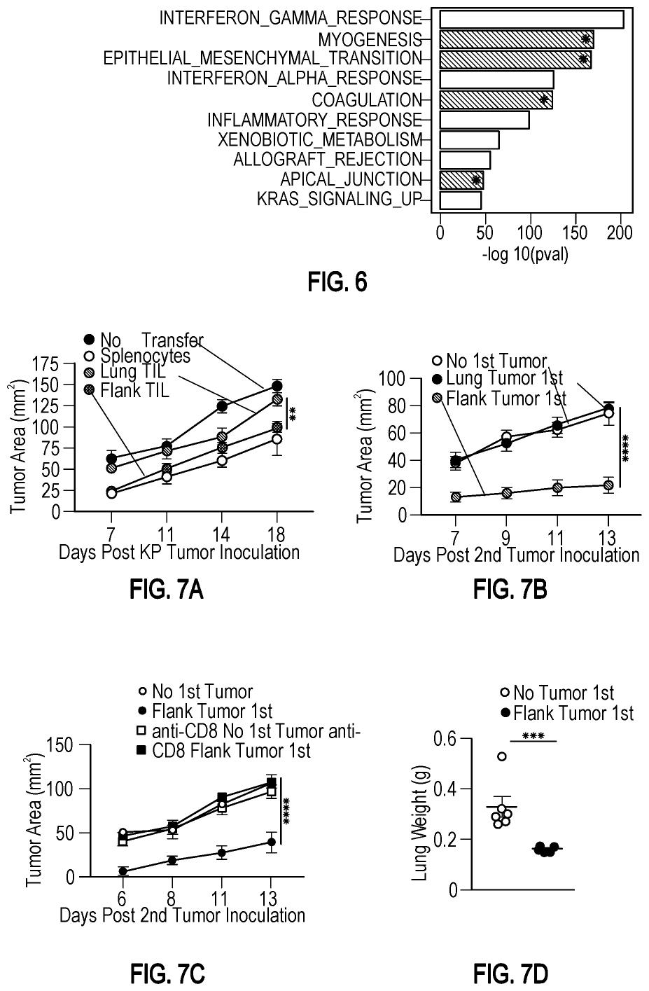

Another possibility was that tumor cell-intrinsic differences, arising from differential seeding or microenvironmental queues of anatomic sites drove different CD8⁺ T cell responses. To determine tumor-cell intrinsic differences, scRNA-seq was performed on tumor cells isolated from lung and flank tumors (data not shown). Differential gene expression analysis determined small set of 175 genes that were differentially regulated between tumor cells isolated from lung or flank tumors, including increased MHC class I expression on tumor cells isolated from the lung (data not shown). Pathway analysis determined that lung tumor cells showed an increased response to interferon when compared to flank tumor cells (FIG. 6), indicative of an ongoing anti-tumor immune response in the lung. These results suggested that the observed differences in CD8⁺ T cell responses between lung and flank tumors were not driven by differential TCR repertoires or tumor cell-intrinsic differences known to impair anti-tumor immunity, but support the notion that CD8⁺ T cells undergo alternate differentiation pathways depending on the anatomic site of tumor growth.

T_{Ldys} is a Persistent T Cell State

The tumor microenvironment can induce gene expression changes in T cells. It was sought to determine whether the dysfunction of lung TIL was dependent on the lung tumor microenvironment for induction and maintenance. Fluorescence activated cell sorted TIL were adoptively transferred from flank or lung tumors into Rag2^{-/-} mice and allowed the TIL to reconstitute the recipient mice. The reconstituted Rag2^{-/-} mice were inoculated with flank KP tumors to assess whether functional differences between lung and flank TIL could be detected when challenged with an equal tumor microenvironment. Flank TIL led to slower tumor growth compared to lung TIL (FIG. 7A), indicating that even when re-challenged with an identical tumor microenvironment outside of the lung, lung TIL retained reduced functionality. This result suggested that once induced, T_{Ldys} is a persistent T cell state that does not require the lung tumor microenvironment for maintenance. To probe for early differences in T cell responses against lung and flank tumors, a concomitant immunity assay was performed. Mice bearing flank or lung KP tumors were challenged with a second KP tumor implanted on the contralateral flank 7 days following the first tumor inoculation. Mice bearing an initial flank tumor experienced significantly improved tumor control of the second tumor compared to naïve mice. Strikingly, mice with an initial lung tumor showed no protection against a second flank tumor (FIG. 7B), consistent with observations of reduced effector molecule expression in lung TIL. Antibody-mediated depletion of CD8⁺ cells confirmed that differences in concomitant immunity were due to differential CD8⁺ T cell responses (FIG. 7C). Finally, to determine whether entry into the lung microenvironment was sufficient to induce lung-specific CD8⁺ T cell dysfunction after initial T cell activation, the effect of an initial flank tumor, capable of inducing a strong effector CD8⁺ T cell response, on the growth of subsequent lung tumors was assessed. Interestingly, the CD8⁺ T cell response induced by an initial flank tumor protected mice against a second lung tumor challenge (FIG. 7D). This result indicated that the lung tumor microenvironment was not directly imposing dysfunction on T cells after tumor infiltration. Together these data suggested that T_{Ldys} is a persistent dysfunctional T cell state established early during the anti-lung tumor immune response.

T_{Ldys} is Established During T Cell Priming

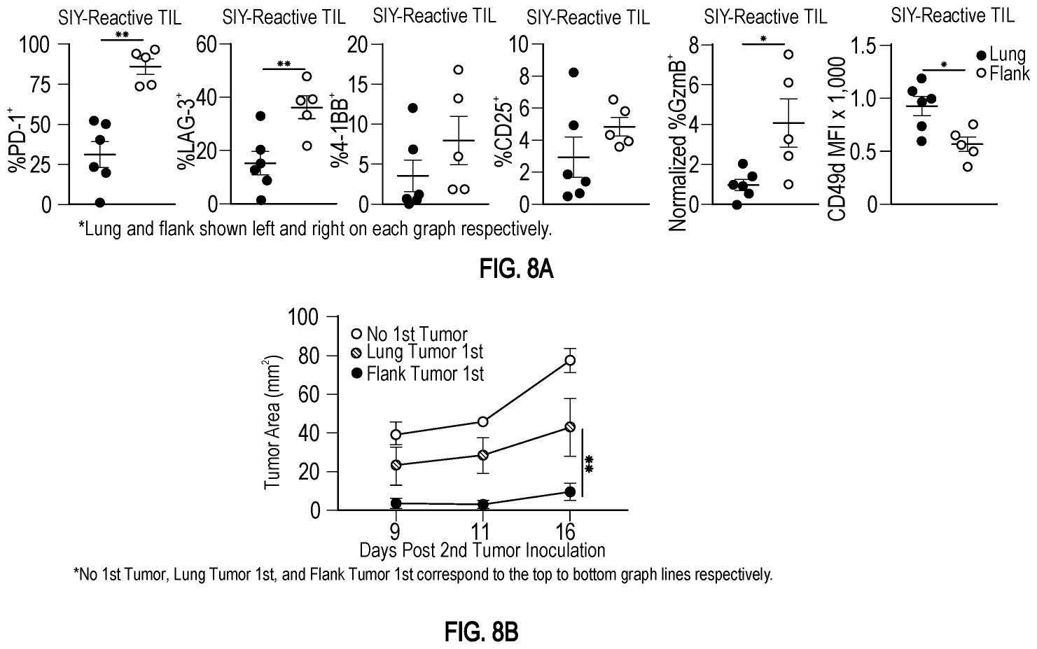

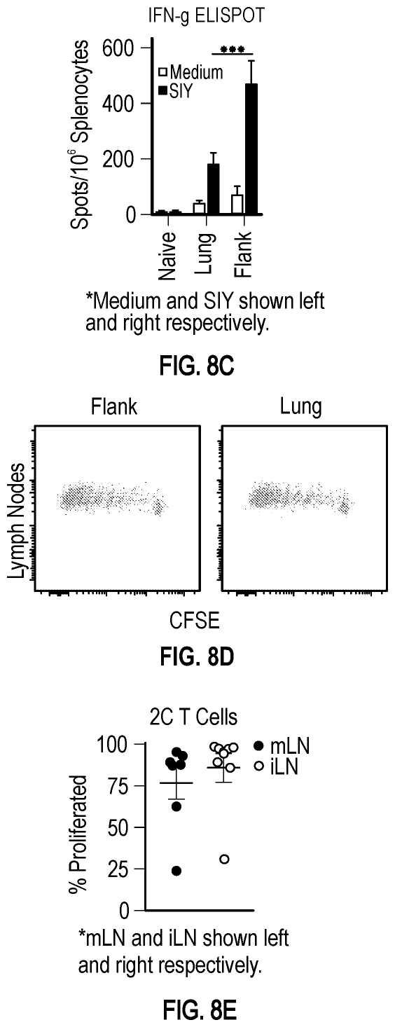

Since differences in effector CD8⁺ T cell function were observed within the first week after tumor inoculation, it was hypothesized that these differences might be established during T cell priming in the tumor-draining lymph node (TdLN). To study anti-tumor CD8⁺ T cell priming in more detail, KP cells were engineered to express the model antigen SIYRYYGL (KP.SIY) (SEQ ID NO. 1). To determine whether the SIY-reactive T cell response resembles the immune response characterized against the parental KP cell line, key molecules were assessed differentially regulated on TIL. Consistent with previous experiments (FIG. 2C), SIY-reactive CD8⁺ T cells from lung tumors had reduced expression of effector and exhaustion molecules, but increased expression of CD49d (FIG. 8A). Further, concomitant immunity following lung KP.SIY tumor inoculation was reduced compared to flank KP.SIY tumors (FIG. 8B). Based on these observations, it was concluded that the model antigen SIY did not affect the observed phenotype of T_{Ldys} in CD8⁺ T cells.

To begin to study CD8⁺ T cell priming an IFN-γ ELISPOT was performed on tumor-bearing mice 7 days post tumor inoculation. KP.SIY lung tumors led to significantly fewer IFN-γ-producing, SIY-reactive splenocytes compared to flank tumors (FIG. 8C), consistent with decreased concomitant immunity after lung tumor challenge (FIG. 7B). To determine whether these differences in CD8⁺ T cell responses against lung and flank tumors were due to a lack of priming in TdLNs, next, an adoptive T cell transfer approach using TCR-transgenic 2C T cells, which are specific for SIY presented on H2-K^{b 24} was utilized. Naïve, CFSE-labeled 2C T cells were transferred into mice with KP.SIY lung or flank tumors on day 7 post tumor inoculation and the degree of T cell activation was determined in TdLN 72h later. Intriguingly, 2C T cells proliferated robustly in both the lung-draining mediastinal LN (mLN) and the flank-draining inguinal LN (iLN) (FIGS. 8D-8E). These data suggested that the degree of activation of tumor-reactive CD8⁺ T cells primed in the mLN and iLN was similar, indicating that the observed functional differences in anti-tumor CD8⁺ T cell responses were likely due to qualitative differences induced during priming.

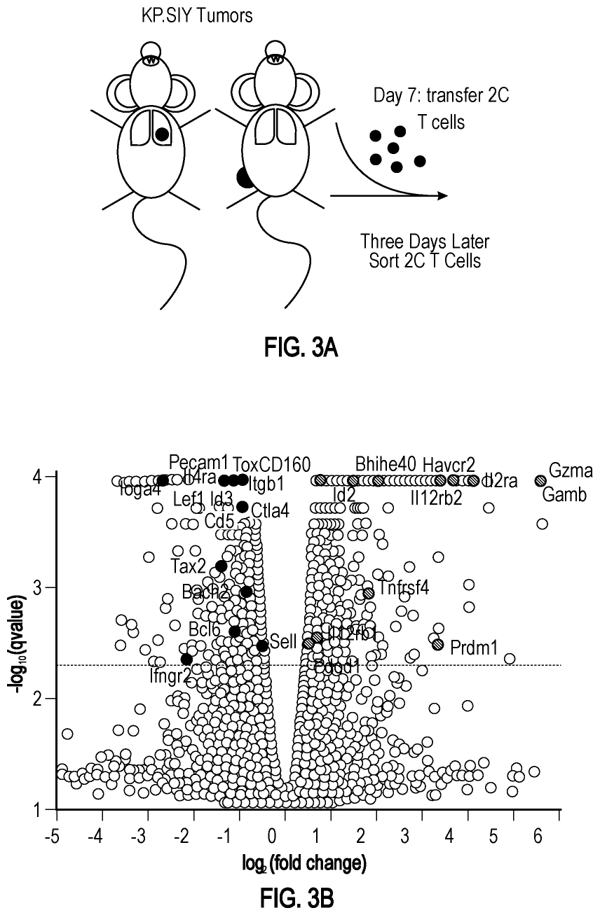

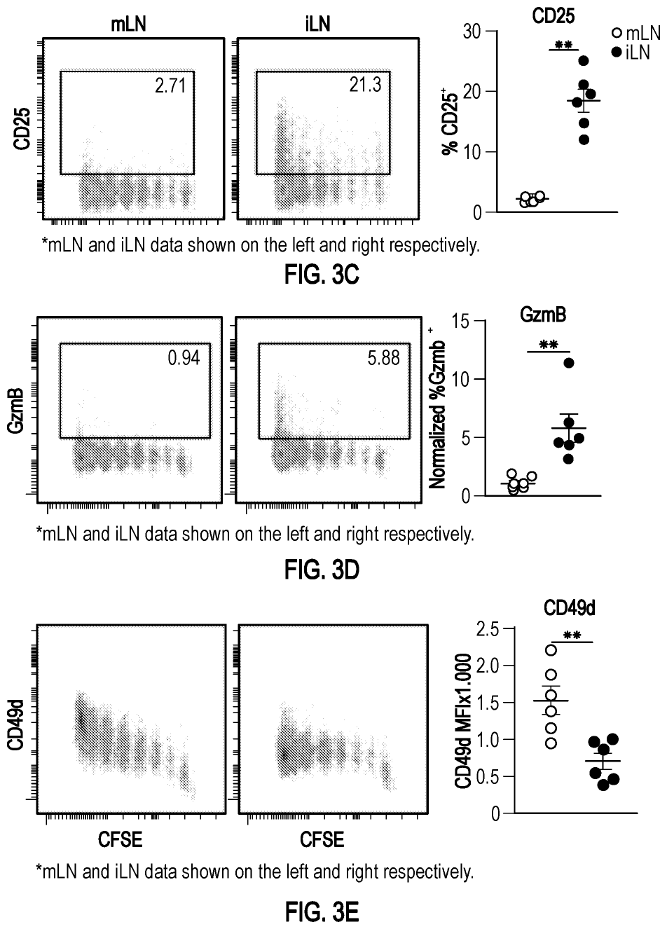

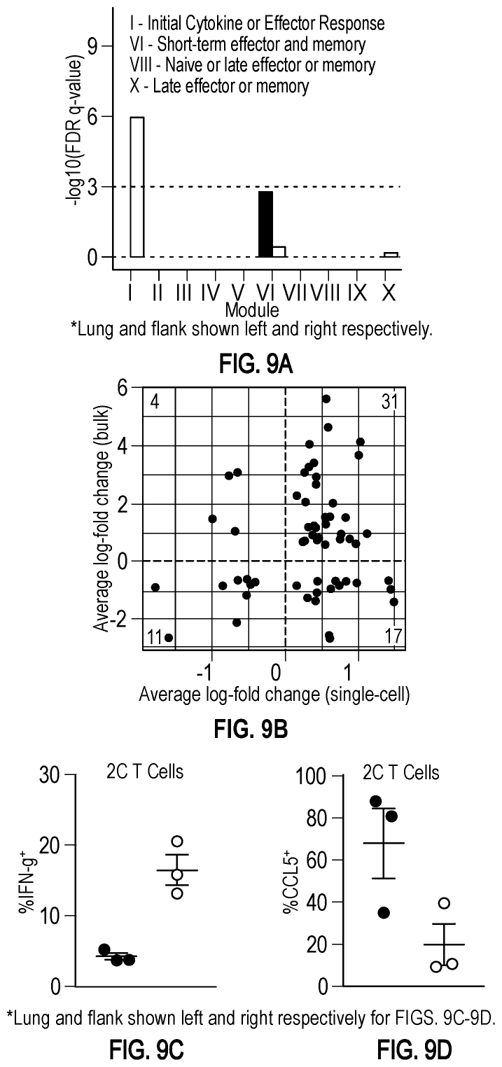

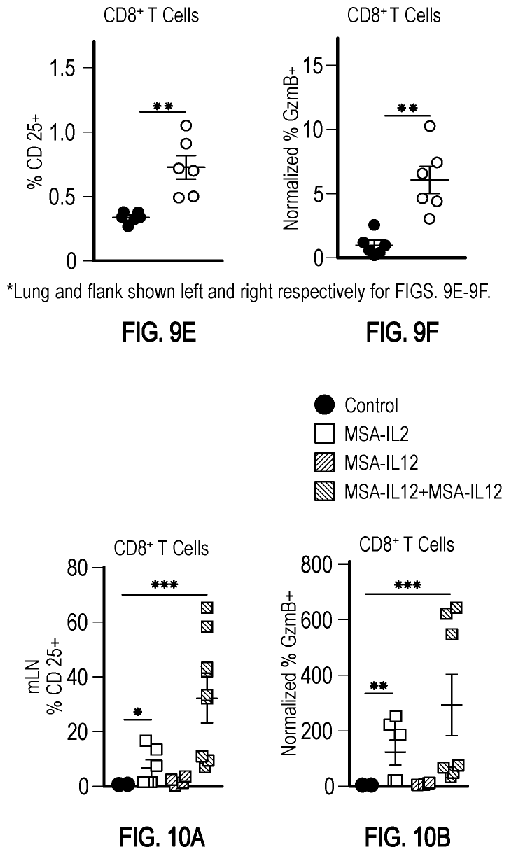

To understand the molecular differences between CD8⁺ T cells activated in response to KP.SIY lung or flank tumors, the above-described approach was used to transfer 2C T cells, along with fluorescence activated cell sorting and Smart-Seq, to determine transcriptional differences between 2C T cells primed in the mLN or iLN (FIG. 3A). 1,744 genes were differentially expressed between iLN and mLN primed 2C T cells. Consistent with the scRNA-seq data of endogenous TIL from flank tumors, 2C T cells primed in iLN showed high transcription levels of genes associated with effector function (Gzma, Gzmb, Il2ra, Il12rb1, Il12rb2, Prdm1, Bhlhe40, and Id2) and exhaustion (Pdcd1, Havcr2, Tnfrsf4, Cd160)¹⁴ (FIG. 3B). In stark contrast, 2C T cells primed in the mLN showed high transcript levels of genes associated with persistence and inhibition of effector T cell differentiation (Sell, Pecarnl, Bcl6, Id3, Lef1, Ctla4, Bach2, Tox, Tox2) and genes associated with lung T cell responses (Ccl5, Itga4, Itgb1)¹⁹⁻²¹ (FIG. 3B). The differential expressed gene signatures were compared to previously described transcriptional profiles of T cell phenotypes¹⁵ and affirmed that CD8⁺ T cells activated in the mLN entered a memory-like T cell phenotype while T cells iLN were enriched for effector differentiation (FIG. 9A). These observations were highly consistent with the data obtained from the TIL analysis (FIG. 2C and provide compelling evidence that the observed qualitative differences in anti-tumor CD8⁺ T cell responses against lung and flank tumors are established during priming in the TdLN. Direct comparison between the recently activated 2C T cells (day 3; KP.SIY) and tumor infiltrating endogenous CD8⁺ T cells (day 14; KP) revealed that 42 genes were consistently regulated between early and late CD8⁺ T cells responses (FIG. 9B and providing further evidence that differences in T cell responses between lung and flank tumors originate from differences that occur during T cell priming in TdLN. Flow cytometry affirmed that the effector molecules CD25, GzmB and IFN-γ were significantly reduced on T cells activated in mLN compared to those from the iLN (FIGS. 3C-3D and FIG. 9C), while mLN-primed 2C T cells showed higher levels of CD49d and CCL5 (FIG. 3E and FIG. 9D). Intriguingly, host CD8⁺ T cells likewise showed higher CD25 and GzmB in the iLN, indicating that differences in CD8⁺ T cell are not restricted to 2C T cells (FIGS. 9E-9F). It was therefore concluded that CD8⁺ T cell priming in the mLN leads to T_{Ldys} responses characterized by impaired anti-tumor CD8⁺ T cell effector differentiation and unresponsiveness to CBT. T_{Ldys} appears to be fundamentally distinct from conventional exhaustion (Tₑₓ), which can be observed in CD8⁺ T cell responses against flank KP tumors induced in the iLN.

T_{Ldys} Dominates a Subset of Patients with NSCLC

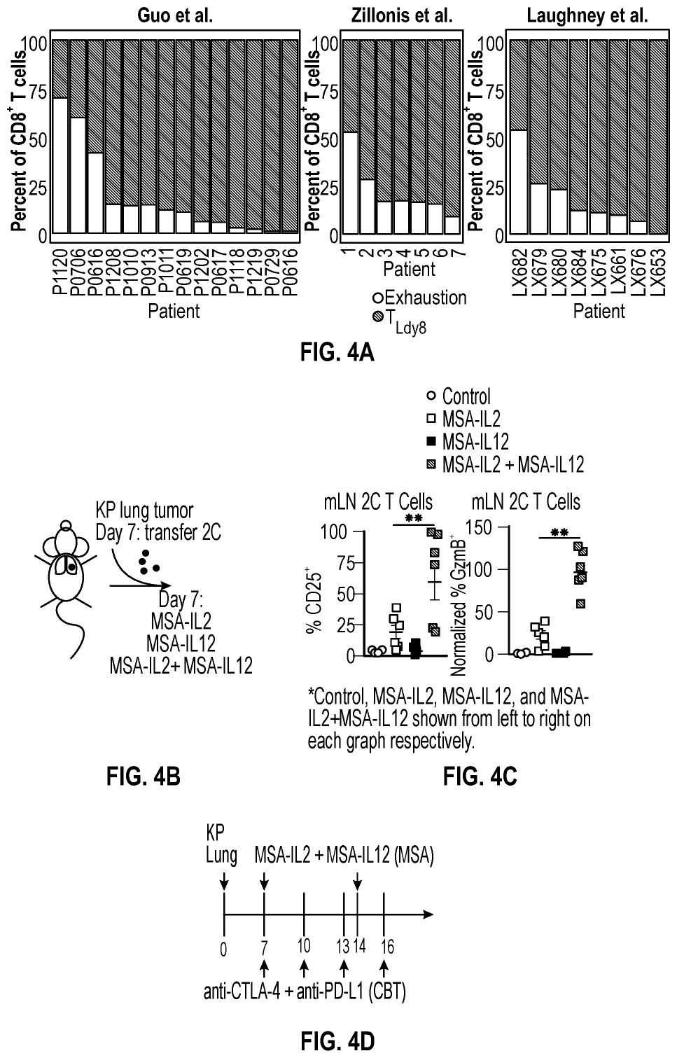

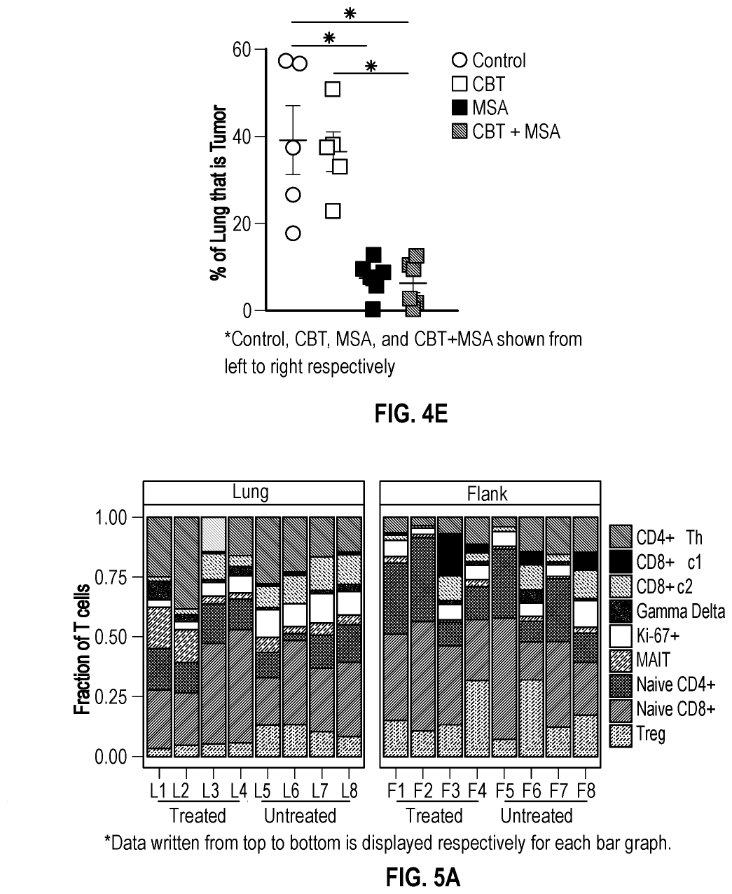

It was then sought to determine whether the T_{Ldys} CD8⁺ T cell state was also present in T cells from patients with NSCLC. Three published scRNA-seq data sets of NSCLC patients 25-27 were examined: the top 50 DEGs from CD8⁺ c1 and CD8⁺ c2 (FIG. 2B) were used to generate exhaustion and T_{Ldys} signatures, respectively, and assessed their expression in tumor-infiltrating CD8⁺ T cells from the human data sets. Strikingly, it was found that across all human NSCLC data sets, the majority of CD8⁺ T cells appeared enriched for the T_{Ldys} signature derived from CD8⁺ c2, while only a fraction of CD8⁺ T cells were enriched for the exhaustion signature derived from CD8+c1 (data not shown). For individual patients, 14 out of 29, or 48% of patients, had TIL with a predominant (>87.5%) T_{Ldys} phenotype (FIG. 4A). Interestingly, comparing gene expression profiles of CD8⁺ T cells among NSCLC data sets and a cutaneous melanoma²⁸ data set, it was found that CD8⁺ T cells infiltrating cutaneous melanomas had increased expression of genes associated with the exhaustion signature while the Tuys signature dominated CD8⁺ T cells from NSCLC patients (Data not shown). These data suggest that CD8⁺ T cells infiltrating human NSCLC can differentiate into the T_{Ldys} phenotype, which is distinct from the conventional exhaustion phenotype, potentially explaining why a fraction of NSCLC patient fails to respond to CBT despite having a T cell infiltrate.

IL-2 and IL-12 Therapy Prevents T_{Ldys}

The data suggested an early induction of the T_{Ldys} program during T cell priming in the mLN. Conversely, bulk RNA-seq analysis indicated significantly higher expression of Il2ra, Il12rb, and Il12rb2 (FIG. 3B) during T cell priming in the iLN in response to flank tumors, suggesting an increased capacity of T cells in the iLN to receive IL-2 and IL-12 signals. Several studies have highlighted the importance of CD25 expression in promoting differentiation to effector and memory phenotypes^{29,30}, while IL-12R signaling can contribute to the upregulation of cytotoxic effector molecules³¹. Both IL-12 and IL-2 can increase CD25 expression thus establishing a positive feedback loop^{32,33}. It was therefore hypothesized that exposure to IL2 and IL12 during T cell priming might overcome T_{Ldys} differentiation to induce effector molecule expression in response to KP lung tumors. To test this hypothesis, 2C T cells were transferred to mice bearing KP.SIY lung tumors and on the same day administered IL-2 and IL-12 fusions to murine serum albumin (MSA-IL2, MSA-IL12)^{34,35} (FIG. 4B). MSA-IL2 slightly increased CD25 and GzmB expression, while MSA-IL12 had no measurable effect on 2C T cells (FIG. 4C). Strikingly, combined administration of MSA-IL2 and MSA-IL12 led to a significant increase in both CD25 and GzmB levels in 2C T cells in mLNs of mice (FIG. 4C). Similar effects were also observed on endogenous T cells, highlighting the broad effect of these treatments on the anti-tumor CD8⁺ T cell response (FIGS. 10A-10B). These data indicate that combined IL-2 and IL-12 signaling are sufficient to overcome inadequate T_{Ldys} differentiation and instead promote effector CD8⁺ T cell differentiation in mLNs.

Cytokine Therapy Plus CBT Controls Lung Tumors

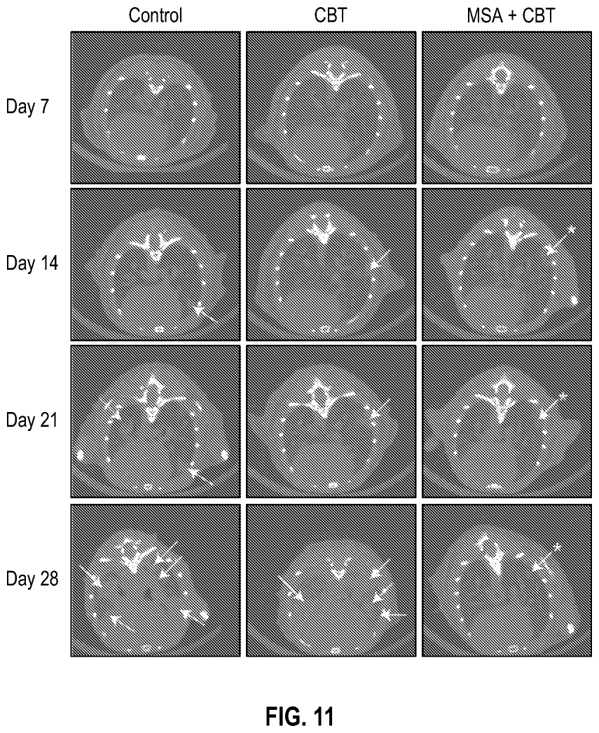

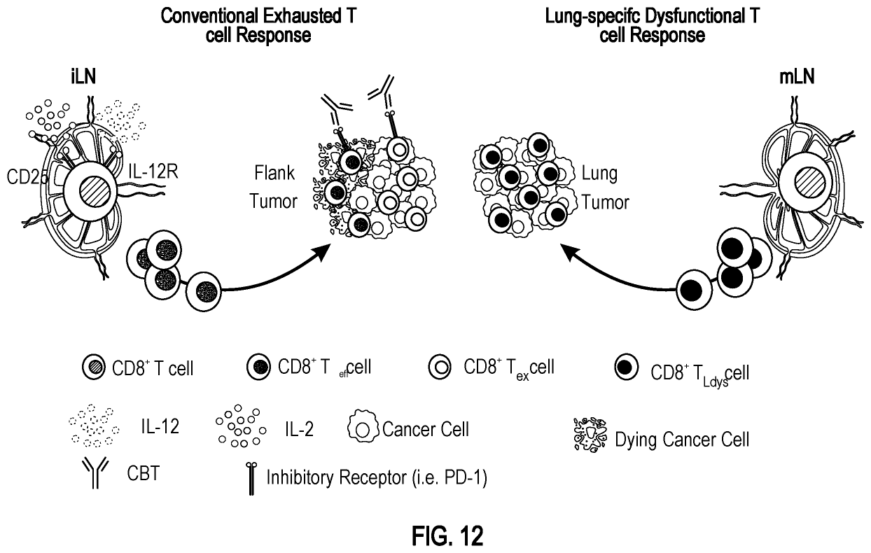

Based on the significant change in T cell differentiation induced by MSA-IL2+MSA-IL12 administration during priming, whether cytokine treatment would synergize with CBT to induce tumor control was assessed next. Mice were inoculated with KP parental lung tumors and treatment with CBT, MSA-IL2+MSA-IL12, or the combination was initiated on day 7 (FIG. 4D). On day 21 mice were euthanized and lungs were analyzed for tumor burden. Consistent with previous results, CBT alone had no effect on tumor growth. Both MSA-IL2+MSA-IL12 (MSA) and the combination of CBT and MSA-IL2+MSA-IL12 (CBT+MSA) induced dramatic reductions in lung KP tumors, however. To confirm these findings, a longitudinal study was conducted using Micro-CT comparing mice left untreated or treated with CBT or CBT+MSA. These longitudinal data provided further evidence that CBT+MSA treatment invigorates protective lung tumor-reactive T cells responses, as reduced tumor size was observed over time in these mice, while CBT only treated mice showed progressive tumor growth (FIG. 11A). In sum, data showed that CD8⁺ T cell responses against lung tumor lesion fail to respond to CBT due to T_{Ldys} differentiation during priming, preventing the acquisition of effector and exhaustion molecules, which can be therapeutically rescued by combined IL-2 and IL-12 therapy.

### Discussion

A novel state of lung-specific T cell dysfunction (Tuy s) that is unresponsive to current CBT agents was identified. A key finding of the study is that T_{Ldys} is induced during T cell priming in the TdLN, after which the activated tumor-reactive T cells fail to acquire effector function and markers of T cell exhaustion (FIGS. 3A-3E). This functionally impaired T cell response is found in the CD8⁺ TIL population, which fails to respond to anti-CTLA-4 and anti-PD-L1 CBT (FIG. 1G). Therapeutically, administering MSA-IL-2 and MSA-IL-12 during priming was sufficient to induce effector T cell differentiation in the mLN (FIG. 4C). The analysis of published patient data sets identifies a related T cell subset in human NSCLC patients which is mutually exclusive with exhausted T cells (FIGS. 4A-4B). Based on these observations, it was posited that patients with a predominantly dysfunctional, T_{Ldys}-like CD8⁺ T cell response fail to respond to CBT, while patients with a sizable fraction of exhausted T cells observe a clinical benefit in response to CBT.

Data suggested responsiveness to CBT is determined during T cell priming in the TdLN, with T cells in CBT-resistant tumors differentiating into a lung-specific dysfunctional (Tuy s) state distinct from conventional T cell exhaustion (Tₑₓ). The inability of CBT to rescue these dysfunctional, but not exhausted, T cells could explain failed responses to immunotherapy in T cell-inflamed tumors, both in NSCLC and other cancer types.

Materials and Methods

Mice.

C57BL/6, CD45.1⁺ and Rag2^{-/-} mice were obtained from Taconic Biosciences. 2C Rag2^{-/-}CD45.1⁺ mice were bred and maintained in house. All mice were housed and bred under specific pathogen free (SPF) conditions at the Koch Institute animal facility. Mice were gender-matched and age-matched to be 6-12 weeks old at the time of experimentation.

Generation of expression vectors.

The pLV-EF1α-IRES-puro vector (Addgene #85132) was digested with BamHI and EcoRI restriction enzymes (NEB) to linearize the vector. The cerulean-—SIY insert was generated using the Cerulean-N1 vector (Addgene #54742) linked to a codon-optimized sequence of the SIYRYYGL (SIY) peptide (SEQ ID NO. 1). The zsgreen insert was generated from pCAGGS_zsGreen_minOVA (a gift from Max Krumme1). The inserts were then cloned into the linearized pLV-EF1α-IRES-puro vector (final constructs referred to as ‘pLV-EF1α-cerulean-SIY-IRES-puro’ and ‘pLV-EF1α-zsgreen-IRES-purs’) using the In-Fusion cloning kit (Takara Bio), amplified, and sequenced for accuracy.

Tumor cell lines and tumor outgrowth studies.

The parental KP NSCLC cell line was a gift from the Jacks laboratory at MIT and validated using Dartmouse SNP analysis. The LL/2 cell line was purchased from the ATCC. The KP tumor line stably expressing cerulean-SIY was generated by lentiviral transduction of the parental tumor line with the pLV-EF1α-cerulean-SIY-IRES-puro construct and puromycin (Gibco) selection. The KP tumor line stably expressing zsgreen was generated by lentiviral transduction of the parental tumor line with the pLV-EF1α-zsgreen-IRES-puro construct and puromycin (Gibco) selection. Expression was periodically assessed using flow cytometry of cerulean-expressing cells.

Tumor cell lines were cultured at 37° C. and 5% CO₂ in DMEM (Gibco) supplemented with 10% FBS (Atlanta Biologicals), 1% penicillin/streptomycin (Gibco), and 1X HEPES (Gibco). Tumor cells were harvested by trypsinization (Gibco) and washed 2 times with 1×PBS (Gibco). Cells were resuspended in PBS, and 2.5×10⁵ tumor cells were injected subcutaneously into the flanks of mice, or intravenously via the tail vein. Subcutaneous tumor area measurements (calculated as length×width) were collected 2-3 times a week with calipers until the endpoint of the study. For lung tumor area measurements, lungs were resected and fixed in 10% neutral buffered formalin (Sigma), then processed and paraffin embedded, sectioned, and H&E stained at the Koch Institute histology core facility. Lung and tumor areas of H&E sections were measured using QuPath software, and the percentage of lung area that was tumor was calculated in excel.

Immunohistochemistry.

Flank tumors and tumor-bearing lungs were fixed in 10% formalin. Fixed tissues were processed, paraffin embedded, sectioned, and stained by the Hope Babette Tang Histology Facility at the Koch Institute at MIT. For anti-CD8 staining, a 1:200 dilution of anti-CD8 (clone 4SM16, eBioscience 14-0195-82) was used.

Tumor dissociation.

Tumor-bearing mice were injected retro-orbitally with fluorescently-labeled anti-CD45 antibodies (CD45-IV) 3 minutes prior to euthanasia to differentiate tumor- and lung-infiltrating immune cells from circulating immune cells. Spleens and lymph nodes were dissected from mice and physically dissociated through a 70 □m filter to generate single cell suspensions. Splenocyte suspensions were lysed with ACK lysis buffer (Gibco) for 3 minutes to deplete red blood cells. Subcutaneous tumors and tumor-bearing lungs were dissected from mice, weighed, and collected in 5 mL., DMEM (Gibco) containing the human tumor dissociation kit (Miltenyi) enzymes. Tumors were dissociated using the gentleMACS Octo Dissociator (Miltenyi). Following the digestion, tumor or lung pieces were mashed through a 70 μm filter with a 1 mL syringe plunger to generate a single cell suspension. The dissociated cells were washed 2 times with PBS and were layered over Ficoll (GE). Cells were spun over Ficoll at 450 g for 30 minutes with the lowest settings of acceleration and brakes. The layer at the interface of Pica and PBS, which contained the majority of the live immune cells, was collected and washed with PBS.

Flow Cytometry and Cell Sorting.

Prior to staining, cells were washed with FACS staining buffer (chilled PBS containing 1% FBS and 2 mM EDTA). Cells were resuspended in 50 uL of the antibody-containing staining buffer, plus eBioscience Fixable Viability Dye eFluor 780 or eFluor 506 to distinguish live and dead cells and with anti-CD16/CD32 (clone 93, BioLegend) to prevent non-specific antibody binding. Cell surface proteins were stained for 20 min on ice with fluorophore-conjugated antibodies at a 1:200 dilution. Cells were then washed twice and resuspended in eBioscience Fixation/Permeabilization buffer and incubated 30 minutes at room temperature. Cells were then washed twice and resuspended in staining buffer with intracellular antibodies. To obtain absolute counts of cells, Precision Count Beads (BioLegend) were added to samples according to manufacturer's instructions. Flow cytometry sample acquisition was performed on a LSR Fortessa cytometer (BD), and the collected data was analyzed using FlowJo v10.5.3 software (TreeStar). For cell sorting, the surface staining was performed as described above under sterile conditions, and cells were acquired and sorted into trizol using a FACSAria III sorter (BD). For TIL analysis, cells were pre-gated on Live, CD45⁺, CD45-IV⁻, TCRbeta⁺, single cells, CD4⁻, CD8⁺, CD62L⁻.

SeqWell sequencing and analysis.

Single-cell suspensions from lung and SQ tumors were prepared as described above. Mice received anti-CD45-PE antibody retro-orbitally 3 minutes prior to euthanasia. Cell suspensions were washed twice with cold PBS+0.5% BSA+2 mM EDTA and then enriched for tumor-resident immune cells by performing negative selection with anti-PE microbeads (Miltenyi Biotec) followed by positive selection with anti-CD45 microbeads (Miltenyi Biotec). Cells were then processed for single-cell RNA sequencing using the Seq-Well platform with second strand chemistry, as previously described^{11,23}. Libraries were barcoded and amplified using the Nextera XT kit (Illumina) and were sequenced on a Novaseq 6000 or a NextSeq 550 (Illumina).

Single-Cell Data Processing and Visualization

Raw read processing of single-cell RNA sequencing reads was performed as previously described³⁸. Briefly, reads were aligned to the mm10 reference genome and collapsed by cell barcode and unique molecular identifier (UMI). Correction for ambient RNA contamination was performed using SoupX v1.2.1. Then, cells with less than 1000 unique genes detected and genes detected in fewer than 5 cells were filtered out, and the data for each cell was log-normalized to account for library size. Genes with log-mean expression values greater than 0.1 and a dispersion of greater than 1 were selected as variable genes, and the ScaleData function in Seurat was used to regress out the number of UMI and percentage of mitochondrial genes in each cell. Principal components analysis was performed. The number of principal components used for visualization was determined by examination of the elbow plot, and two-dimensional embeddings were generated using uniform manifold approximation and projection (UMAP). Clusters were determined using Louvain clustering, as implemented in the FindClusters function in Seurat, and clusters that contained T cells were selected for further analysis. These cells were reprocessed with the same processing and clustering steps described above. Differential gene expression was performed for each cluster, and clusters corresponding to similar phenotypes were merged.

Pathway Enrichment Analysis

Gene sets representing relevant pathways were obtained from MSigDB^{39,40}. A score for the expression of genes in each pathway was calculated for each cell using the AddModuleScore function in Seurat⁴¹. Pathways in the hallmarks gene collection were used to analyze CD8⁺ T cells and KP.zsGreen tumor cells.

Paired Single-Cell TCR Sequencing

Paired TCR sequencing and read alignment was performed as previously described⁴². Briefly, whole-transcriptome amplification product from each single-cell library was enriched for TCR transcripts using biotinylated Tcrb and Tcra probes and magnetic streptavidin beads. The enrichment product was further amplified using V-region primers and Nextera sequencing handles, and the resulting libraries were sequenced on an Illumina Miseq or NextSeq 550. CDR3 consensus sequences were then aligned as outlined previously. Only sequences with a CDR3 consensus frequency greater than 0.6 were used for analysis. TCR specificity groups were determined by clustering all Tcrb CDR3 sequences according to Levenshtein distance using complete-linkage hierarchical clustering and cutting the resulting dendrogram at a height of one.

Adoptive TIL transfer.

Flank and lung tumors were dissociated and prepared for FACS as described above. TIL were identified as Live, CD45⁺, non-circulating Thyl⁺ cells. Recipient RAG2^{-/-} mice received equal numbers of either lung or flank TIL intravenously. TIL were allowed 12+ weeks to reconstitute RAG2^{-/-} mice, after which the reconstituted mice were inoculated with flank tumors.

Concomitant immunity assay.

Mice were injected with KP tumors subcutaneously or intravenously as outlined above. Seven days post tumor injection, mice were injected with a second KP tumor on the contralateral flank, or intravenously.

CD8 antibody depletion.

Mice were injected with 200 μg of anti-CD8 antibody (clone 53-6.7 Bio X Cell) weekly for the duration of the experiment, beginning 48 hours before tumor inoculation.

IFNγ-ELISpot.

ELISpot plates (EMD Millipore) were coated overnight at 4° C. with anti-IFNγ (BD Biosciences). Plates were washed and blocked with DMEM supplemented with 10% EBS, 1% penicillin/streptomycin, and 1X HUES for 2 hr at room temperature (RT). Spleens were harvested from mice at day 5 or day 7 post-tumor inoculation and mashed through a 70 μm filter with a 1 mL: syringe plunger to generate a single cell suspension. Red blood cells were lysed with 500 μL of ACK Lysing Buffer (Gibco) on ice for 2 min. Splenocytes were washed 3 times with chilled PBS and 1×10⁵ cells were assayed per well in the presence or absence of 160 nM STY peptide or a mixture of 100 ng/mL PMA (Sigma-Aldrich) and 1 μg/mL ionomycin (Sigma-Aldrich) as a positive control. Plates were developed the next day using the BD mouse IFNγ-ELISpot kit, following manufacturer's protocol.

2C T cell adoptive transfer.

Spleens and inguinal lymph nodes of 2C RAG2^{-/-} CD45.1⁺ mice were dissected and made into single cell suspensions as described above. Cells were labeled with CFSE or cell trace violet (Life Technologies) following the manufacturer's instructions. Approximately 1 million labeled cells were transferred to mice KP.SIY tumor bearing seven days post tumor inoculation. Recipient animals were euthanized and analyzed three days post 2C T cell transfer.

Smart-seq bulk RNA sequencing and analysis.

2C T cells were adoptively transferred to tumor-bearing mice as described above. 72 hours post adoptive transfer, TdLN cells were isolated as described above, and prepared for FACS sorting as described above. 2C T cells were identified as Live, CD45⁺, Thy 1⁺, single cells, CD4⁻, CD8⁺, CD45.1^{+ and were FACS-sorted directly into Trizol reagent. RNA was extracted from sorted cells with a Trizol—chloroform extraction. The isolated RNA-containing aqueous layer was passed over an RNeasy column (Qiagen) following the manufacturer's instructions to obtain purified RNA in elution buffer. RNA was frozen at -}80 Celsius until used in RNA sequencing. RNA library preparation and sequencing were performed. Sequencing was performed using the HiSeq 2000 sequencing system (Illumina). Sequence alignment and .bam files were sorted using Samtools. Differential gene expression analysis was performed on the sorted .bam files with Cufflinks.

Analysis of Public Datasets

Human NSCLC datasets were downloaded from the Gene Expression Omnibus (GEO) database, under accession numbers GSE99254²⁵, GSE127465⁴³ and GSE123902²⁶. After filtering expression matrices based on the number of unique genes detected in each cell, the processing pipeline described above was utilized to identify clusters corresponding to CD8⁺ T cells. These clusters were then analyzed separately.

CBT Treatment

Mice were injected intraperitoneally with anti-CTLA-4 (clone UC10-4F10-11, Bio X Cell) and anti-PD-L1 (clone 10F.9G2, Bio X Cell) antibodies on days 7, 10, 13, and 16 post-tumor inoculation. Each mouse received 100 μg of each antibody per treatment.

MSA-IL2 and MSA-IL12 Generation

MSA-cytokine fusions were generated as previously described^{34,35.}

MSA Treatment

Each mouse was injected retro-orbitally with 5.94E-10 mol/mouse MSA-IL2 and/or 1.42E-11 mol/mouse MSA-IL12 per treatment. For experiments with 2C T cell adoptive transfer, mice received one dose of MSA-cytokine fusions on day 7 post-tumor inoculation and were analyzed on day 10 post-tumor inoculation. For therapeutic experiments, mice were dosed with MSA-IL2 and/or MSA-IL12 on days 7 and 14 post-tumor inoculation and analyzed on day 21 post-tumor inoculation.

μCt

A Bruker Skyscan 1276 was used to acquire a series of images with a rotation step of 0.65 degrees over a 360-degree rotation. Images were acquired with x-ray tube settings of 100 kV, 200 μA, and an exposure time of 90 ms with a 0.5 mm aluminum beam filter. 4×4 detector binning was used for an isotropic resolution of 40.16 μm. Anesthesia was induced at 3% isoflurane and maintained at 2.0-2.5% during imaging, which lasted 76 seconds. Image reconstruction was performed used the Bruker NRecon software.

Statistical analysis.

Statistical analyses were performed using GraphPad Prism (GraphPad) and R. All data are shown as mean±SEM. For flow cytometry, immunohistochemistry, and tumor outgrowth studies, statistical analyses were performed with Mann-Whitney U (MWU) test for comparisons of two groups or two-way ANOVA for multiple comparisons over time, with *p<0.05; **p<0.01; ***p<0.001; ****p<0.0001. % For differential gene expression between T cell clusters, p-values were calculated using a two-sided Wilcoxon rank-sum test and were corrected with Bonferroni correction. For comparison of gene modules to previously published T cell states, FDR q-values were calculated using a one-tailed hypergeometric test and were corrected with Bonferroni correction. For pathway analysis, p-values were calculated using a two-tailed Wilcoxon rank-sum test and were corrected with Bonferroni correction.

Sequence List:

| SEQ ID NO | Sequence |
|---|---|
| 1 | Ser Ile Tyr Arg Tyr Tyr Gly Leu |
| 2 | Cys Ala Ser Ser Thr Pro Gly Gln Gly Asn Glu Arg Leu Phe Phe |
| 3 | Cys Ala Ser Gly Gly Thr Gly Gly Gln Asp Thr Gln Tyr Phe |
| 4 | Cys Ala Ser Ser Phe Ser Gly Gly Ala Ala Glu Gln Phe Phe |
| 5 | Cys Ala Ser Ser Phe Thr Gly Val Gly Asn Thr Leu Tyr Phe |
| 6 | Cys Ala Ser Ser Gly Gly Gly Leu Ala Glu Thr Leu Tyr Phe |
| 7 | Cys Ala Ser Ser Leu Gly Gly Arg Ala Glu Gln Phe Phe |
| 8 | Cys Ala Ser Ser Leu Gly Val Asn Asn Gln Ala Pro Leu Phe |
| 9 | Cys Ala Ser Ser His Gly Gly Thr Ser Ala Glu Thr Leu Tyr Phe |
| 10 | Cys Ala Ser Gly Asp Pro Gly Leu Gly Val Gly Glu Gln Phe Phe |
| 11 | Cys Ala Ser Ser Asp Gly Gln Gly Ala Ser Glu Thr Leu Tyr Phe |
| 12 | Cys Ala Ser Ser Tyr Arg Gly Arg Glu Gln Tyr Phe |
| 13 | Cys Ala Trp Ser Leu Pro Trp Gly Ala Gln Asn Thr Leu Tyr Phe |
| 14 | Cys Ala Ser Ser Leu Arg Gly Arg Glu Gln Tyr Phe |
| 15 | Cys Ala Ser Ser Ala Asp Arg Gly Lys Asn Thr Leu Tyr Phe |
| 16 | Cys Ala Ser Gly Glu Thr Asn Ser Gly Asn Thr Leu Tyr Phe |
| 17 | Cys Ala Ser Ser Phe Thr Gly Tyr Ala Glu Gln Phe Phe |
| 18 | Cys Ala Ser Lys Gly Gly Gly Glu Asp Thr Gln Tyr Phe |
| 19 | Cys Ala Ser Arg Pro Gly Thr Gly Ser Tyr Glu Gln Tyr Phe |
| 20 | Cys Ala Ser Ser Asp Gly Asn Tyr Ala Glu Gln Phe Phe |
| 21 | Cys Ala Ser Ser Glu Asp Gln Asn Thr Leu Tyr Phe |
| 22 | Cys Ala Ser Ser Leu Gly Gly Ala Asn Ser Pro Leu Tyr Phe |
| 23 | Cys Ala Ser Ser Ser Gly Gln Asn Thr Glu Val Phe Phe |
| 24 | Cys Ala Ser Ser Ile Thr Gly Lys Glu Gly Gln Asn Thr Leu Tyr Phe |
| 25 | Cys Ala Arg Asp Arg Glu Asp Gln Ala Pro Leu Phe |
| 26 | Cys Ala Ser Ser Arg Trp Gly Gln Asp Thr Gln Tyr Phe |
| 27 | Cys Ser Ala Gly Thr Gly Gln Asn Thr Gly Gln Leu Tyr Phe |
| 28 | Cys Ala Ser Ala Gly Asp Ser Tyr Glu Gln Tyr Phe |
| 29 | Cys Ala Ser Ser Leu Thr Ala Asn Ser Asp Tyr Thr Phe Gly Ser Gly |
| 30 | Cys Ala Ser Gly Asp Ala Arg Val Glu Asp Thr Gln Tyr Phe |
| 31 | Cys Ala Ile Gln Asn Ser Gly Asn Thr Leu Tyr Phe |
| 32 | Cys Ala Ser Ser Pro Gly Leu Gly Gly Ala Glu Thr Leu Tyr Phe |
| 33 | Cys Ala Ser Ser Ser Thr Gly Gly Ala Gly Glu Gln Tyr Phe |
| 34 | Cys Ala Ser Asn Pro Leu Asn Ser Gly Asn Thr Leu Tyr Phe |
| 35 | Cys Ala Ser Leu Asp Trp Ser Gln Asn Thr Leu Tyr Phe |
| 36 | Cys Ala Ser Ser Thr Pro His Arg Gly Ser Gln Asn Thr Leu Tyr Phe |
| 37 | Cys Ala Ser Ser Ile Thr Glu Val Phe Phe |
| 38 | Cys Ala Ser Ser Leu Gly Gly Cys Asp Tyr Thr Phe Gly Ser Val Thr |
| | Arg Leu |
| 39 | Cys Ala Ser Ser Leu Gly Asn Leu Asn Thr Gly Gln Leu Tyr Phe |
| 40 | Cys Ala Ser Ser Phe Pro Ser Ala Asn Thr Gly Gln Leu Tyr Phe |
| 41 | Cys Ala Ser Ser Leu Glu Thr Gly Gly Thr Tyr Glu Gln Tyr Phe |
| 42 | Cys Ala Trp Arg Gly Thr Gly Val Ala Glu Thr Leu Tyr Phe |
| 43 | Cys Ala Ser Gly Asp Gln Asn Tyr Ala Glu Gln Phe Phe |
| 44 | Cys Ala Ser Ser Arg Asp Arg Val Tyr Glu Gln Tyr Phe |
| 45 | Cys Ala Ser Gly Asp Ala Gly Arg Gly Gly Asn Thr Leu Tyr Phe |
| 46 | Cys Ala Ser Ser Leu Glu Gly Gly Asn Thr Glu Val Phe Phe |
| 47 | Cys Ala Ser Ser Leu Asp Gly Gly Gln Glu Gln Tyr Phe |
| 48 | Cys Ala Ser Ser Phe Gly Gly Gly Thr Gly Gln Leu Tyr Phe |
| 49 | Cys Ala Ser Ser Leu Gly Gln Ala Asn Thr Glu Val Phe Phe |
| 50 | Cys Ala Ser Arg Val Gln Gly Gly Ala Glu Thr Leu Tyr Phe |
| 51 | Cys Ala Ser Ser Ala Arg Gly Leu Glu Asp Thr Gln Tyr Phe |
| 52 | Cys Ala Ser Ser Pro Thr Gly Phe Ala Glu Gln Phe Phe |
| 53 | Cys Ala Ser Ser Phe Trp Gly Glu Asp Thr Gln Tyr Phe |
| 54 | Cys Ala Ser Ser Ile Ser Gly Asp Tyr Thr Phe Gly Ser Val Thr Arg |
| 55 | Cys Ala Ser Ser Leu Ala Arg Arg Ser Gly Asn Thr Leu Tyr Phe |
| 56 | Cys Ala Ser Ser Phe Gly Leu Asn Ser Asp Tyr Thr Phe Gly Ser Val |
| | Thr Arg Leu |
| 57 | Cys Ala Ser Ser Glu Gly Asp Gln Asn Thr Leu Tyr Phe |

### REFERENCES

1 Tumeh, P. C. et al. PD-1 blockade induces responses by inhibiting adaptive immune resistance. Nature 515, 568-571, doi:10.1038/nature13954 (2014).
2 Rafei, H., El-Bahesh, E., Finianos, A., Nassereddine, S. & Tabbara, I. Immune-based Therapies for Non-small Cell Lung Cancer. Anticancer Res 37, 377-387, doi:10.21873/anticanres.11330 (2017).
3 Gajewski, T. F., Schreiber, H. & Fu, Y. X. Innate and adaptive immune cells in the tumor microenvironment. Nat Immunol 14, 1014-1022, doi:10.1038/ni.2703 (2013).
4 Thommen, D. S. & Schumacher, T. N. T Cell Dysfunction in Cancer. Cancer Cell 33, 547-562, doi:10.1016/j.cce11.2018.03.012 (2018).
5 Wherry, E. J. & Kurachi, M. Molecular and cellular insights into T cell exhaustion. Nat Rev Immunol 15, 486-499, doi:10.1038/nri3862 (2015).
6 Hodi, F. S. et al. Improved survival with ipilimumab in patients with metastatic melanoma. N Engl J Med 363, 711-723, doi:10.1056/NEJMoa1003466 (2010).
7 Topalian, S. L. et al. Safety, activity, and immune correlates of anti-PD-1 antibody in cancer. N Engl J Med 366, 2443-2454, doi:10.1056/NEJMoa1200690 (2012).
8 Spranger, S. et al. Density of immunogenic antigens does not explain the presence or absence of the T-cell-inflamed tumor microenvironment in melanoma. Proc Natl Acad Sci USA 113, E7759-E7768, doi:10.1073/pnas.1609376113 (2016).
9 LaFave, L. M. et al. Epigenomic State Transitions Characterize Tumor Progression in Mouse Lung Adenocarcinoma. Cancer Cell 38, 212-228e213, doi: 10.1016/j.cce11.2020.06.006 (2020).
10 Spranger, S. et al. Mechanism of tumor rejection with doublets of CTLA-4, PD-1/PD-L1, or IDO blockade involves restored IL-2 production and proliferation of CD8(+) T cells directly within the tumor microenvironment. J Immunother Cancer 2, 3, doi:10.1186/2051-1426-2-3 (2014).
11 Gierahn, T. M. et al. Seq-Well: portable, low-cost RNA sequencing of single cells at high throughput. Nat Methods 14, 395-398, doi:10.1038/nmeth.4179 (2017).
12 Gubin, M. M. et al. Checkpoint blockade cancer immunotherapy targets tumour-specific mutant antigens. Nature 515, 577-581, doi:10.1038/nature13988 (2014).
13 Matsushita, H. et al. Cancer exome analysis reveals a T-cell-dependent mechanism of cancer immunoediting. Nature 482, 400-404, doi:10.1038/nature10755 (2012).
14 Williams, J. B. et al. The EGR2 targets LAG-3 and 4-1BB describe and regulate dysfunctional antigen-specific CD8⁺ T cells in the tumor microenvironment. J Exp Med 214, 381-400, doi:10.1084/jem.20160485 (2017).
15 Best, J. A. et al. Transcriptional insights into the CD8(+) T cell response to infection and memory T cell formation. Nat Immunol 14, 404-412, doi:10.1038/ni.2536 (2013).
16 Skon, C. N. et al. Transcriptional downregulation of S1pr1 is required for the establishment of resident memory CD8⁺ T cells. Nat Immunol 14, 1285-1293, doi:10.1038/ni.2745 (2013).
17 Weinreich, M. A. et al. KLF2 transcription-factor deficiency in T cells results in unrestrained cytokine production and upregulation of bystander chemokine receptors. Immunity 31, 122-130, doi: 10.1016/j.immuni.2009.05.011 (2009).
18 Harari, A., Bellutti Enders, F., Cellerai, C., Bart, P. A. & Pantaleo, G. Distinct profiles of cytotoxic granules in memory CD8 T cells correlate with function, differentiation stage, and antigen exposure. J Virol 83, 2862-2871, doi:10.1128/JVI.02528-08 (2009).
19 Galkina, E. et al. Preferential migration of effector CD8⁺ T cells into the interstitium of the normal lung. J Clin Invest 115, 3473-3483, doi:10.1172/JCI24482 (2005).
20 Grau, M. et al. Antigen-Induced but Not Innate Memory CD8 T Cells Express NKG2D and Are Recruited to the Lung Parenchyma upon Viral Infection. J Immunol 200, 3635-3646, doi:10.4049/jimmuno1.1701698 (2018).
21 Ray, S. J. et al. The collagen binding alpha1beta1 integrin VLA-1 regulates CD8 T cell-mediated immune protection against heterologous influenza infection. Immunity 20, 167-179, doi: 10.1016/s1074-7613 (04)⁰⁰⁰²¹-4 (2004).
22 Honda, T. et al. Tuning of antigen sensitivity by T cell receptor-dependent negative feedback controls T cell effector function in inflamed tissues. Immunity 40, 235-247, doi:10.1016/j.immuni.2013.11.017 (2014).
23 o, T. K. et al. Second-Strand Synthesis-Based Massively Parallel scRNA-Seq Reveals Cellular States and Molecular Features of Human Inflammatory Skin Pathologies. Immunity 53, 878-894 e877, doi:10.1016/j.immuni.2020.09.015 (2020).
24 Stegall, M. D. et al. The 2C T-cell transgenic mouse: an in vivo model of allospecific cytotoxic T-cell activation and homing. Transplant Proc 31, 779, doi:10.1016/s0041-1345(98)⁰¹⁷⁶¹-8 (1999).
25 Guo, X. et al. Global characterization of T cells in non-small-cell lung cancer by single-cell sequencing. Nat Med 24, 978-985, doi:10.1038/s41591-018-0045-3 (2018).
26 Laughney, A. M. et al. Regenerative lineages and immune-mediated pruning in lung cancer metastasis. Nat Med 26, 259-269, doi:10.1038/s41591-019-0750-6 (2020).
27 Zilionis, R. et al. Single-Cell Transcriptomics of Human and Mouse Lung Cancers Reveals Conserved Myeloid Populations across Individuals and Species. Immunity 50, 1317-1334 e1310, doi:10.1016/j.immuni.2019.03.009 (2019).
28 Tirosh, I. et al. Dissecting the multicellular ecosystem of metastatic melanoma by single-cell RNA-seq. Science 352, 189-196, doi:10.1126/science.aad0501 (2016).
29 Kalia, V. et al. Prolonged interleukin-2Ralpha expression on virus-specific CD8⁺ T cells favors terminal-effector differentiation in vivo. Immunity 32, 91-103, doi:10.1016/j.immuni.2009.11.010 (2010).
30 Pipkin, M. E. et al. Interleukin-2 and inflammation induce distinct transcriptional programs that promote the differentiation of effector cytolytic T cells. Immunity 32, 79-90, doi:10.1016/j.immuni.2009.11.012 (2010).
31 Trinchieri, G. Interleukin-12 and the regulation of innate resistance and adaptive immunity. Nat Rev Immunol 3, 133-146, doi:10.1038/nri1001 (2003).
32 Nguyen, T., Wang, R. & Russell, J. H. IL-12 enhances IL-2 function by inducing CD25 expression through a p38 mitogen-activated protein kinase pathway. Eur J Immunol 30, 1445-1452, doi:10.1002/(SICI)¹⁵²¹-4141(200005)30:5<1445::AID-IMMU1445>3.0.00;2-M (2000).
33 Siegel, J. P., Sharon, M., Smith, P. L. & Leonard, W. J. The IL-2 receptor beta chain (p70): role in mediating signals for LAK, NK, and proliferative activities. Science 238, 75-78, doi:10.1126/science.3116668 (1987).
34 Momin, N. et al. Anchoring of intratumorally administered cytokines to collagen safely potentiates systemic cancer immunotherapy. Sci Transl Med 11, doi:10.1126/scitranslmed.aaw2614 (2019).
35 Zhu, E. F. et al. Synergistic innate and adaptive immune response to combination immunotherapy with anti-tumor antigen antibodies and extended serum half-life IL-2. Cancer Cell 27, 489-501, doi:10.1016/j.cce11.2015.03.004 (2015).
36 Ganesan, A. P. et al. Tissue-resident memory features are linked to the magnitude of cytotoxic T cell responses in human lung cancer. Nat Immunol 18, 940-950, doi:10.1038/ni.3775 (2017).
37 Thommen, D. S. et al. A transcriptionally and functionally distinct PD-1(+) CD8(+) T cell pool with predictive potential in non-small-cell lung cancer treated with PD-1 blockade. Nat Med 24, 994-1004, doi:10.1038/s41591-018-0057-z (2018).
38 Macosko, E. Z. et al. Highly Parallel Genome-wide Expression Profiling of Individual Cells Using Nanoliter Droplets. Cell 161, 1202-1214, doi:10.1016/j.cell.2015.05.002 (2015).
39 Mootha, V. K. et al. PGC-lalpha-responsive genes involved in oxidative phosphorylation are coordinately downregulated in human diabetes. Nat Genet 34, 267-273, doi:10.1038/ng1180 (2003).
40 Subramanian, A. et al. Gene set enrichment analysis: a knowledge-based approach for interpreting genome-wide expression profiles. Proc Natl Acad Sci USA 102, 15545-15550, doi:10.1073/pnas.0506580102 (2005).
41 Butler, A., Hoffman, P., Smibert, P., Papalexi, E. & Satija, R. Integrating single-cell transcriptomic data across different conditions, technologies, and species. Nat Biotechnol 36, 411-420, doi:10.1038/nbt.4096 (2018).
42 Tu, A. A. et al. TCR sequencing paired with massively parallel 3’ RNA-seq reveals clonotypic T cell signatures. Nat Immunol 20, 1692-1699, doi:10.1038/s41590-019-0544-5 (2019).
43 Zilionis, R. et al. Single-Cell Transcriptomics of Human and Mouse Lung Cancers Reveals Conserved Myeloid Populations across Individuals and Species. Immunity 50, 1317-1334 e1310, doi:10.1016/j.immuni.2019.03.009 (2019).

What is claimed is:

1. A method for identifying a T cell having a dysfunctional property, comprising isolating a T cell and determining the presence of effector molecules associated with the T cell, wherein when the T cell expresses high levels of CD49d and CCL5, and TOX in the absence of TIM3 the T cell is a dysfunctional CD8⁺ T (T_{dys}) cell.

2. The method of claim 1, wherein the presence of effector molecules is determined by flow cytometry.

3. The method of claim 1, wherein the T cells are not associated with or have low levels of effector molecules CD25, GzmB and IFN-γ.

4. The method of claim 1, wherein the T cells express high levels of genes associated with persistence and inhibition of effector T cell differentiation.

5. The method of claim 4, wherein the genes associated with persistence and inhibition of effector T cell differentiation are Sell, Pecam1, Bc16, Id3, Lef1, Ctla4, Bach2, Tox, and Tox2.

6. The method of claim 1, wherein the T cells express high levels of genes associated with lung T cell responses.

7. The method of claim 6, wherein the genes associated with lung T cell responses are Cc15, Itga4, Itgb1.

8. The method of claim 1, wherein the T cells express low levels or no genes associated with effector function and exhaustion.

9. The method of claim 8, wherein the genes associated with effector function are Gzma, Gzmb, Il2ra, Il12rb1, Il12rb2, Prdm1, Bhlhe40, and Id2 and exhaustion are Pdcd1, Havcr2, Tnfrsf4, and Cd160.

10. A composition comprising an isolated population of dysfunctional CD8⁺ T (T_{dys}) cells, wherein the T cells express high levels of CD49d and CCL5 and TOX in the absence of TIM3 and are not associated with or have low levels of effector molecules CD25, GzmB and IFN-γ.

11. The composition of claim 10, wherein the isolated population of T_{dys} cells are identified according to a method of claim 1.

12. The composition of claim 10, wherein at least 80% of the cells in the population are T_{dys} cells.

13. A method for treating a subject having cancer, comprising
isolating a sample containing T cells from the subject, determining whether the sample contains dysfunctional CD8⁺ T (T_{dys}) cells, wherein if the sample contains T_{dys} cells then the subject is treated with a checkpoint based enhancing therapy and wherein if the sample does not contain T_{dys} cells then the subject is treated with a checkpoint based therapy (CBT).

14. The method of claim 13, wherein the T_{dys} are T_{Ldys}.

15. The method of claim 14, wherein the cancer is a lung cancer.

16. The method of claim 13, wherein the cells in the sample are identified as T_{dys} cells according to a method of claim 1.

17. A method for treating a subject having cancer, comprising
administering to the subject a checkpoint based enhancing therapy, wherein the checkpoint based enhancing therapy is a compound that disrupts the activity of Tdys cells and administering to the subject a checkpoint based therapy (CBT) in an effective amount to treat the cancer.

18. The method of claim 17, wherein the compound that disrupts the activity of Tdys cells is a cytokine.

19. The method of claim 17, wherein the compound that disrupts the activity of Tdys cells is a promoter of the IL2 pathway.

20. The method of claim 17, wherein the compound that disrupts the activity of Tdys cells is a promoter of the IL12 pathway.

21. The method of claim 17, wherein the compound that disrupts the activity of Tdys cells is a promoter of the IL2 and IL12 pathway.

22. The method of any one of claims 13-21, wherein the CBT is an inhibitory checkpoint antibody.

23. The method of claim 22, wherein inhibitory checkpoint antibody is selected from the group consisting of anti-PD1, anti-PD-L1, anti-CTLA4 therapy.

24. A tumor cell stably expressing cerulean-SIY.

25. The tumor cell of claim 24, wherein the cerulean-SIY comprises SIYRYYGL (SEQ ID NO. 1).

26. The tumor cell of claim 24, wherein the cell comprises pLV-EF1α-cerulean-SIY-IRES-puro.
